(19) United States
(12) Patent Application Publication
(10) Pub. No.: US 2016/0152560 A1
(43) Pub. Date: Jun. 2, 2016

(54) Compounds for the treatment of hypertension and hypertensive end stage renal disease

(71) Applicant: THE REGENTS OF THE UNIVERSITY OF CALIFORNIA, Oakland, CA (US)

(72) Inventors: Igor F. Tsigelny, San Diego, CA (US); Daniel T. O'Connor, San Diego, CA (US); Valentina L. Kouznetsova, San Diego, CA (US); Nilima Biswas, San Diego, CA (US)

(21) Appl. No.: 14/893,802

(22) PCT Filed: May 28, 2014
(86) PCT No.: PCT/US14/39814

§ 371 Date: Nov. 24, 2015

Related U.S. Application Data
(60) Provisional application No. 61/828,083, filed on May 28, 2013.

(51) Int. Cl. C07C 281/18; C07C 335/20; C07D 233/26

(52) U.S. Cl. CPC C07C 281/18; C07D 233/26; C07C 335/20

(57) ABSTRACT

This disclosure relates to compounds and compositions useful for the treatment of hypertension and hypertensive renal disease.

### CROSS-REFERENCE TO RELATED APPLICATIONS

This application claims priority to U.S. Provisional Application Ser. No. 61/828,083, filed on May 28, 2013, which is incorporated herein by reference in its entirety.

### FEDERALLY SPONSORED RESEARCH OR DEVELOPMENT

This invention was made with Government support under Grant No. HL58120, DK awarded by the National Institutes of Health. The Government has certain rights in the invention.

### TECHNICAL FIELD

This disclosure relates to compounds and compositions useful for the treatment of hypertension and hypertensive end stage renal disease.

### BACKGROUND

Hypertension is the most common and lethal of cardiovascular risk factors, yet despite pharmacological advances, it remains only partially controlled by antihypertensive medications. The past 30 years have seen a remarkable rise in the number of people living with end-stage renal disease (ESRD) within the United States, from a population of about 40,000 in 1978 to more than 400,000 in the early 21st century. Increasing incidence of ESRD has been especially evident among African Americans, who, despite constituting only approximately 12% of the general population of the U.S., represent greater than 30% of patients on chronic dialysis.

Chromogranin A (CHGA, OMIM 118910), is the 48 kDa protein found in catecholamine secretory vesicles of chromaffin cells and postganglionic sympathetic axons. CHGA contains characteristic sites for proteolytic cleavage by which it is transformed to biologically active peptides: pancreastatin (hCHGA₂₅₀₋₃₀₁)], prochromacin (bCHGA₇₉₋₄₃₁), vasostatin (hCHGA₁₋₇₆), and catestatin (CST: bovine CHGA₃₄₄₋₃₆₄: RSMRLSFRARGYGFRGPGLQL; human CHGA₃₅₂₋₃₇₂: SSMKLSFRARGYGFRGPGPQL), a well characterized inhibitor of catecholamine release working as antagonist at neuronal nicotinic acetylcholine receptors. In human patients with hereditary hypertension, or their offspring, the concentration of CST in the plasma is diminished, suggesting that its deficiency can play a pathogenic role in development of hypertension. Targeted ablation of the CHGA locus in the mouse results in unbridled hypertension that can be “rescued” by administration of CHGA's catecholamine release-inhibitory catestatin fragment. The catestatin fragment of CHGA exerts both antihypertensive and vasodilatory actions in vivo, in both rodents and humans.

### SUMMARY

Provided herein are compounds which are useful for the treatment of hypertension and hypertensive end stage renal disease (ESRD).

Compound provided herein include compounds of Formula (I):

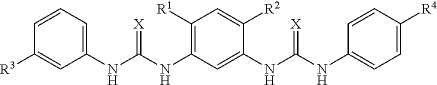

or a pharmaceutically acceptable salt thereof,
wherein:
each X is independently selected from the group consisting of: O and S;
R¹ and R² are independently selected from the group consisting of: H, (C₁-C₆)alkyl, and —C(O)NH(C₁-C₆)alkyl;
R³ is selected from the group consisting of:

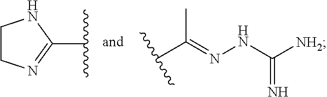

and
R⁴ is selected from the group consisting of:

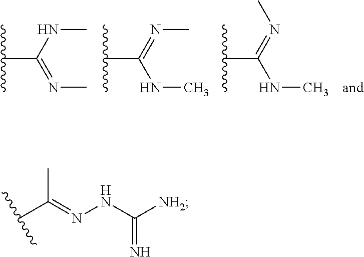

wherein R³ and R⁴ are not the same.

In some embodiments, R₁ and R₂ are independently selected from the group consisting of: H, CH₃, and —C(O)NH(CH₃). In some embodiments, X is O.

In some embodiments, R³ is selected from the group consisting of:

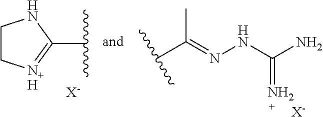

wherein X⁻ is a counteranion. In some embodiments, R³ is

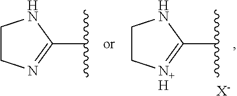

wherein X⁻ is a counteranion.

In some embodiments, R⁴ is selected from the group consisting of:

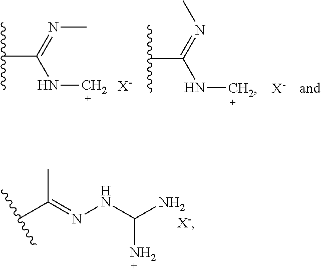

wherein X⁻ is a counteranion. In some embodiments, R⁴ is selected from the group consisting of:

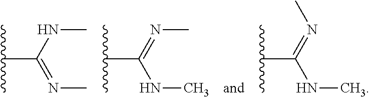

In some embodiments, R⁴ is selected from the group consisting of:

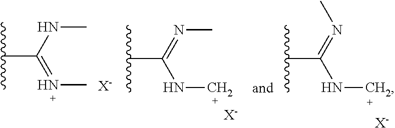

wherein X⁻ is a counteranion.

Non-limiting examples of a compound of Formula (I) include:

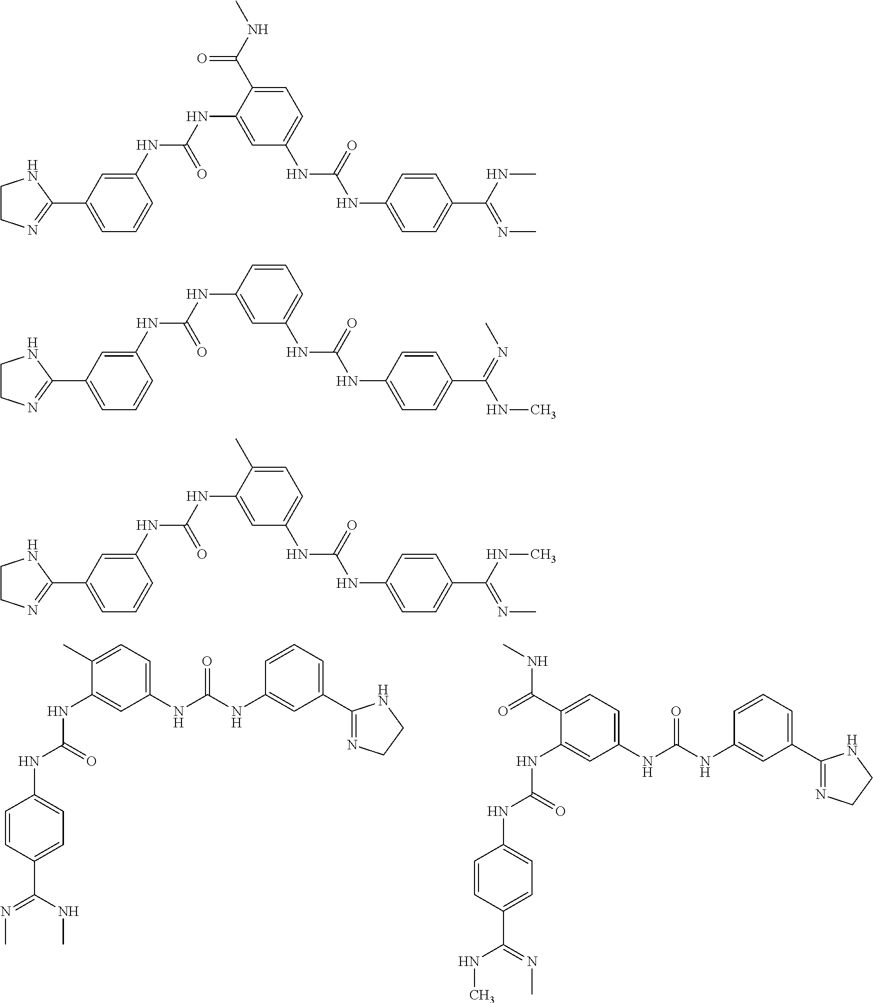

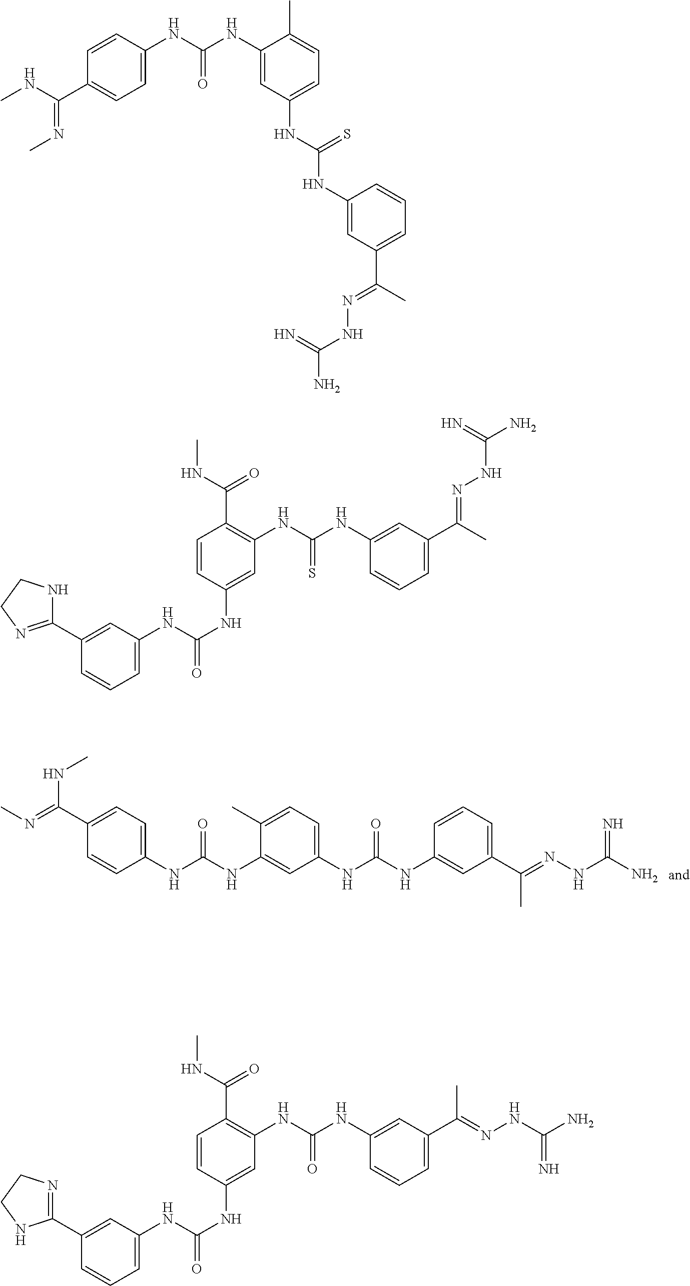

or a pharmaceutically acceptable salt form thereof.

For example, a compound of Formula (I) can be selected from:

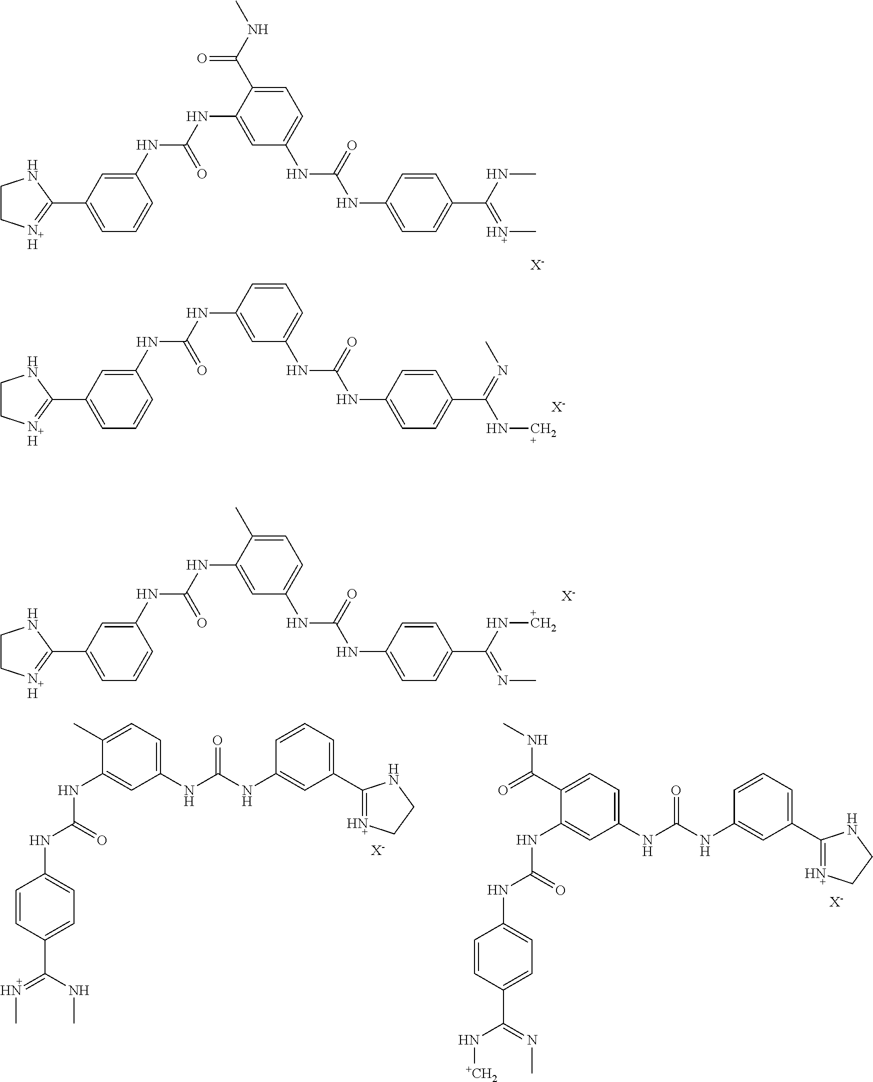

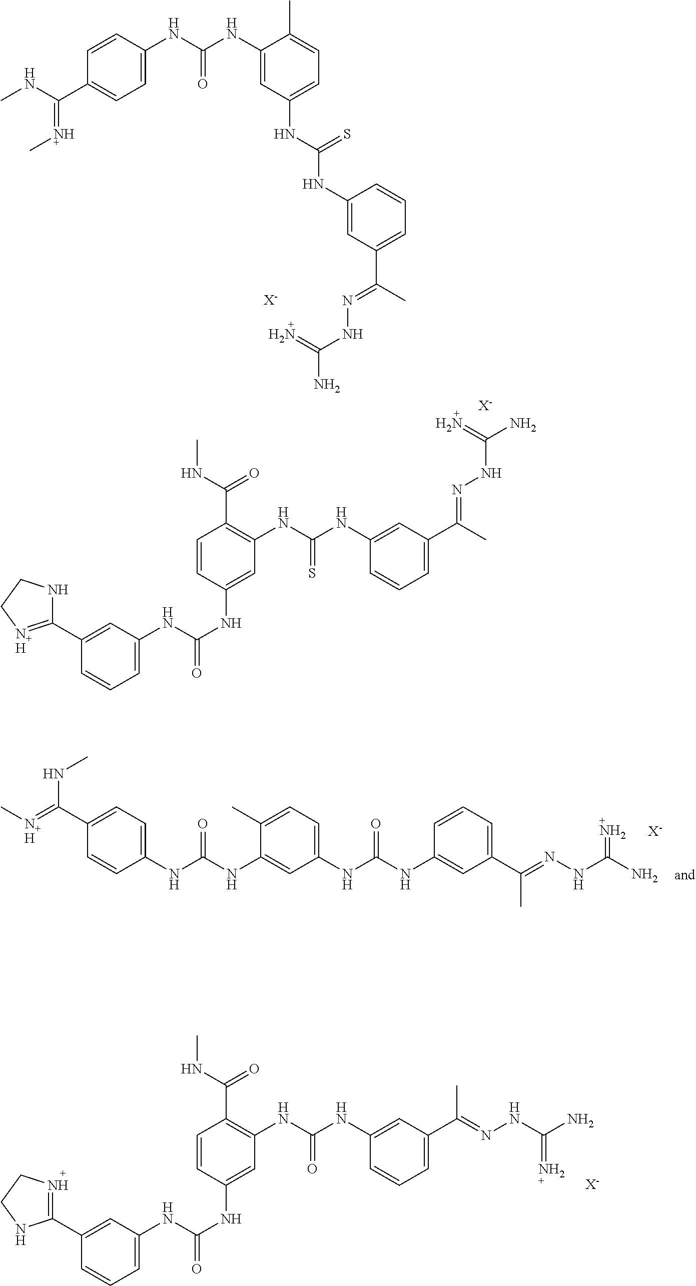

wherein each X⁻ is independently one or more counteranions.

Also provided herein are compounds Formula (II):

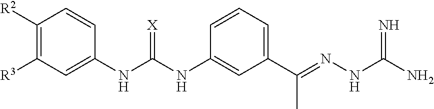

or a pharmaceutically acceptable salt thereof,
wherein:
X is selected from the group consisting of O and S;
R¹ is selected from the group consisting of: H and

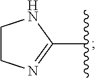 [SMILES: *C1=NCCN1]

R² is selected from the group consisting of: H,

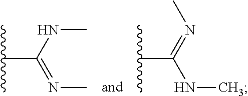

wherein no more than one of R¹ and R² is H.

In some embodiments, a compound of Formula (II) is a compound of Formula (IIA):

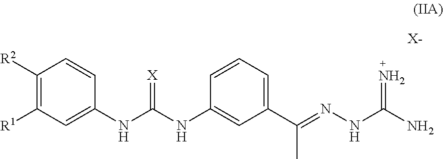

or a pharmaceutically acceptable salt thereof, wherein X⁻ is a counteranion.

In some embodiments, X is S. In some embodiments, X is O.

In some embodiments, R¹ is selected from the group consisting of: H and

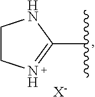

wherein X⁻ is a counteranion.

In some embodiments, R² is selected from the group consisting of: H,

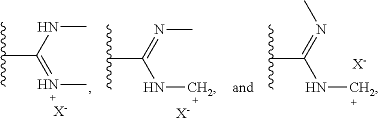

wherein X⁻ is a counteranion.

In some embodiments, R¹ is

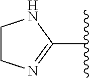 [SMILES: *C1=NCCN1]

and R² is H. In some embodiments, R¹ is

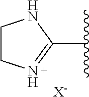

and R² is H, wherein X⁻ is a counteranion. In some embodiments, R¹ is H and

### R² is

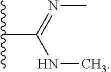 [SMILES: */C(=N/C)NC]

In some embodiments, R¹ is H and R² is

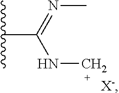

wherein X⁻ is a counteranion.

Non-limiting examples of a compound of Formula (II) include:

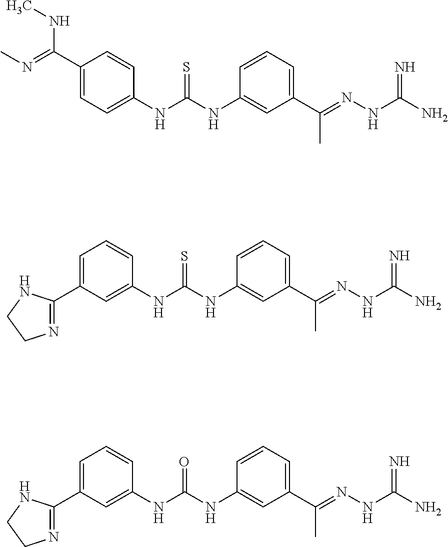 [SMILES: C/N=C(\NC)c1ccc(NC(=S)Nc2cccc(/C(C)=N/NC(=N)N)c2)cc1 ; C/C(=N\NC(=N)N)c1cccc(NC(=S)Nc2cccc(C3=NCCN3)c2)c1 ; C/C(=N\NC(=N)N)c1cccc(NC(=O)Nc2cccc(C3=NCCN3)c2)c1]

or a pharmaceutically acceptable salt thereof.

For example, a compound of Formula (II) can be selected from:

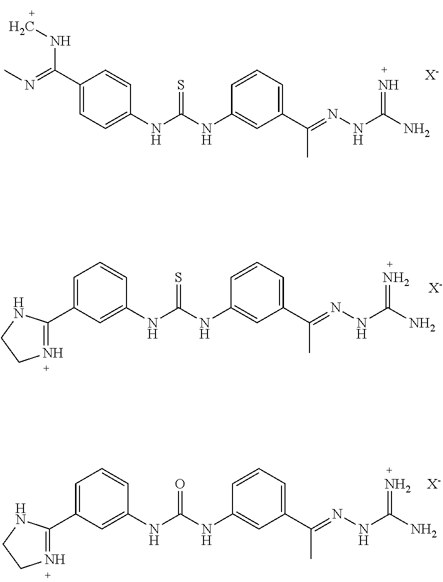

wherein each X⁻ is independently one or more counteranions.

Further provided herein are compound selected from the group consisting of:

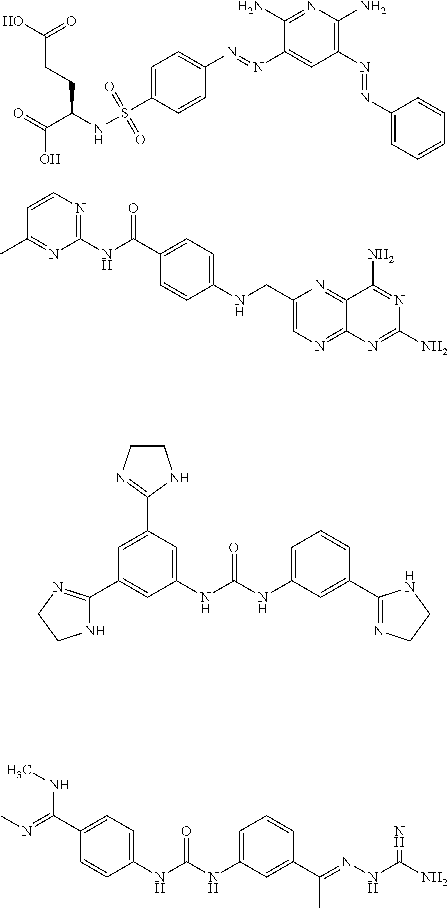

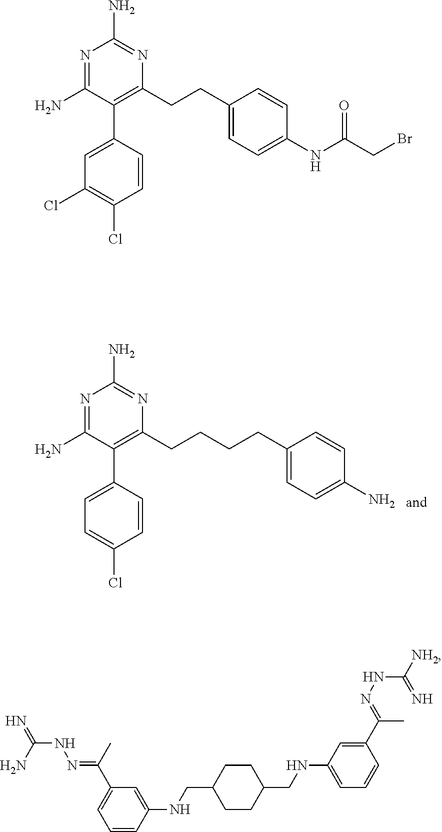 [SMILES: Nc1nc(N)c(-c2ccc(Cl)c(Cl)c2)c(CCc2ccc(NC(=O)CBr)cc2)n1 ; Nc1ccc(CCCCc2nc(N)nc(N)c2-c2ccc(Cl)cc2)cc1 ; C/C(=N\NC(=N)N)c1cccc(NCC2CCC(CNc3cccc(/C(C)=N/NC(=N)N)c3)CC2)c1]

or a pharmaceutically acceptable salt thereof.

In some embodiments, the compound is:

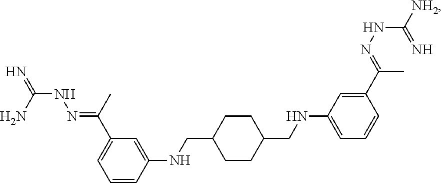 [SMILES: C/C(=N\NC(=N)N)c1cccc(NCC2CCC(CNc3cccc(/C(C)=N/NC(=N)N)c3)CC2)c1]

or a pharmaceutically acceptable salt thereof.

Further provided herein is a pharmaceutical composition comprising a compound provided herein, or a pharmaceutically acceptable salt thereof, and a pharmaceutically acceptable carrier.

The compounds provided herein are also useful for treating hypertension or hypertensive end stage renal disease (ESRD) in a patient in need thereof. In some embodiments, the method includes administering to the patient a therapeutically effective amount of a compound provided herein, or a pharmaceutically acceptable salt thereof.

This disclosure also provides for methods for treating hypertension or hypertensive end stage renal disease (ESRD) in a patient in need thereof. The method can include administering to the patient a therapeutically effective amount of a compound selected from the group consisting of:

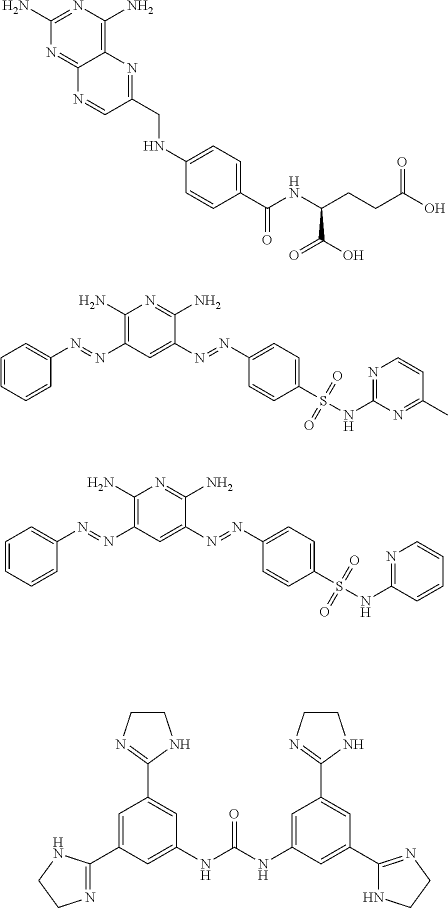 [SMILES: Nc1nc(N)c2nc(CNc3ccc(C(=O)N[C@@H](CCC(=O)O)C(=O)O)cc3)cnc2n1 ; Cc1ccnc(NS(=O)(=O)c2ccc(/N=N/c3cc(/N=N/c4ccccc4)c(N)nc3N)cc2)n1 ; Nc1nc(N)c(/N=N/c2ccc(S(=O)(=O)Nc3ccccn3)cc2)cc1/N=N/c1ccccc1 ; O=C(Nc1cc(C2=NCCN2)cc(C2=NCCN2)c1)Nc1cc(C2=NCCN2)cc(C2=NCCN2)c1]

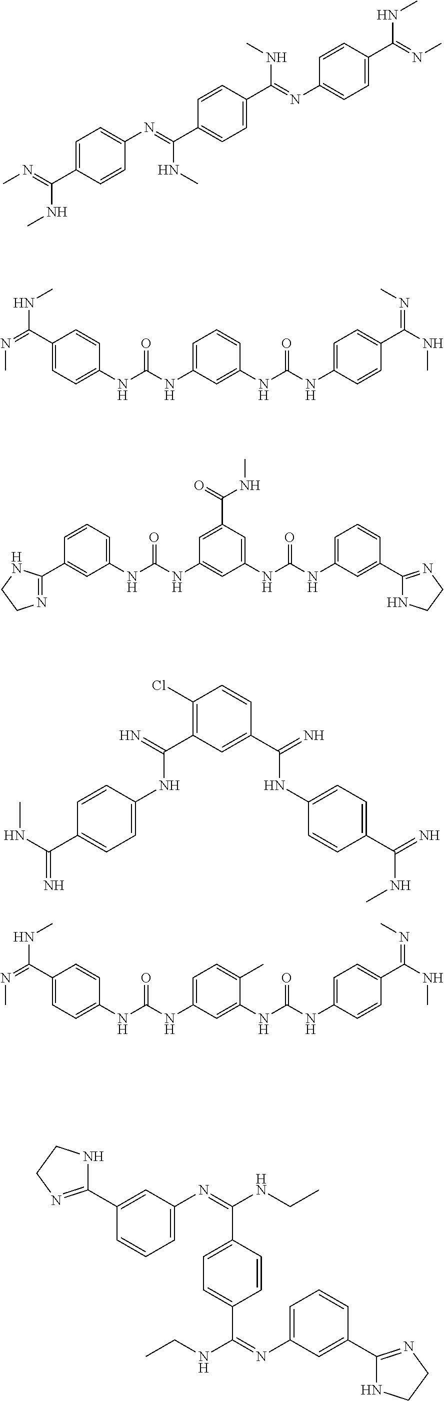

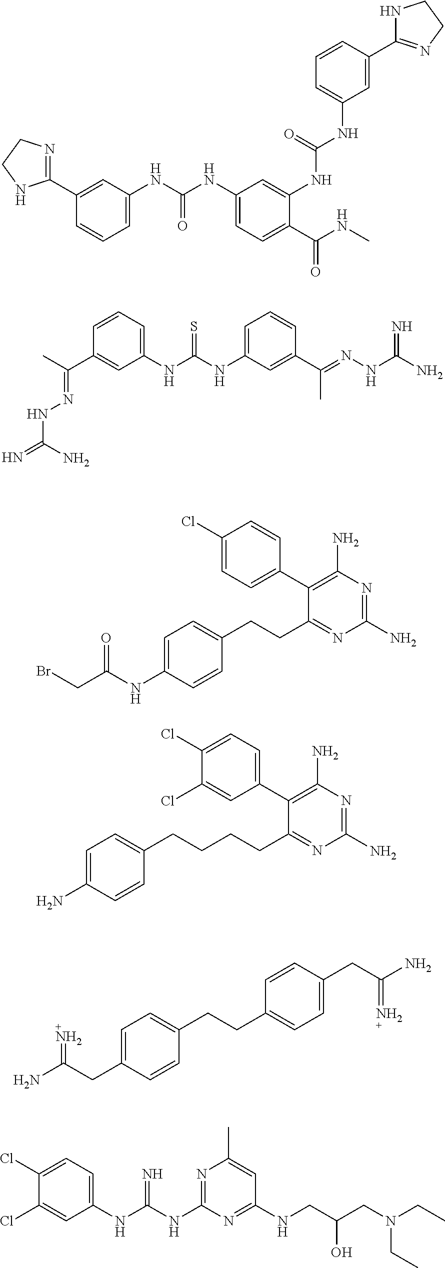

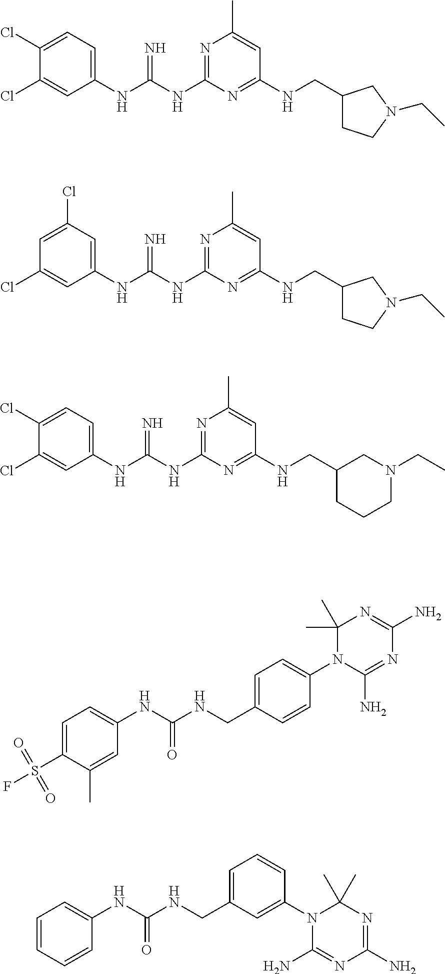 [SMILES: CCN1CCC(CNc2cc(C)nc(NC(=N)Nc3ccc(Cl)c(Cl)c3)n2)C1 ; CCN1CCC(CNc2cc(C)nc(NC(=N)Nc3cc(Cl)cc(Cl)c3)n2)C1 ; CCN1CCCC(CNc2cc(C)nc(NC(=N)Nc3ccc(Cl)c(Cl)c3)n2)C1 ; Cc1cc(NC(=O)NCc2ccc(N3C(N)=NC(N)=NC3(C)C)cc2)ccc1S(=O)(=O)F ; CC1(C)N=C(N)N=C(N)N1c1cccc(CNC(=O)Nc2ccccc2)c1]

or a pharmaceutically acceptable salt thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All appendices, publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF THE DRAWINGS

FIG. 1 shows the primary structure (amino acid sequence) of catestatin with pharmacophore annotation points (purple dots) and pharmacophore features (green and dark blue circles). Annotation points correspond to hydrophobic Leu5, Phe7, and Phe14; and positively charged Arg8, Arg10, and Arg15. Green circles represent hydrophobic and aromatic/hydrophobic features, while dark blue circles represent NCN+ groups/cations/H-bond donors.

FIGS. 2A and 2B illustrate exemplary results of inhibition of secretion testing for cooperativity of test compounds: Hill plots are shown for compounds TKO-10-5 and TKO-10-18. The plots assess the fractional effect of ascending doses of compound to inhibit catecholamine release triggered by 30 μM nicotine from PC12 cells. The slope is the Hill coefficient representing the cooperativity of binding, where a slope=1 indicates noncooperativity (independent action of different antagonist molecules).

FIG. 3A illustrates exemplary results of basal secretion assays. This remained unaltered in the presence of compounds TKO-10-5 or TKO-10-14. FIG. 3B shows that the inhibitory effect of test compounds on catecholamine secretion is specific for nicotinic cholinergic stimulation. Results were analyzed by one-way ANOVA evaluating the effect of compound on nicotine-stimulated norepinephrine secretion (F=2311, p<0.0001). FIG. 3C illustrates that test compounds act as noncompetitive nicotinic cholinergic antagonists. Results were analyzed by one-way ANOVA evaluating the effect of compounds on nicotine at 10 μM (F=32.1, p=0.009), 30 μM (F=158.2, p=0.0009), 60 μM (F=1338.8, p<0.0001), 100 μM (F=7903.9, p<0.0001), or 1000 μM (F=24.6, p=0.01).

FIG. 4. illustrates exemplary effects of cluster-B test compounds (TKO-10-4, TKO-10-5, TKO-10-7, and TKO-10-18) on nicotine-induced uptake of ⁴⁵Ca²⁺ in PC12 cells. Results were analyzed by two-way ANOVA evaluating the effect of compound (F=6.7, p=0.0009), dose (F=1578.9, p<0.0001) and compound-by-dose interaction (F=1.9, p=0.05). The IC₅₀ values (μM) were 0.07 (TKO-10-4), 0.1 (TKO-10-5), 0.1 (TKO-10-7), and 0.09 (TKO-10-18).

FIG. 5 illustrates rescue from elevated SBP (FIG. 5A) and DBP (FIG. 5B) by test compound TKO-10-18; SBP and DBP were monitored by telemetry before and after administration of test compound in BPH/2J mice (compound TKO-10-18, 5 μg/g body weight, by intraperitoneal injection, n=7). FIG. 5C shows the change in heart rate after administration of test compound TKO-10-18 in hypertensive BPH/2J mice. Results are analyzed by two-way, repeated-measures ANOVA, evaluating the effects of drug, time, and time-by-drug interaction.

### DETAILED DESCRIPTION

### Definitions

It is appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

Unless defined otherwise, all technical and scientific terms used herein have the same meaning as is commonly understood by one of ordinary skill in the art to which this disclosure belongs. All patents, applications, published applications, and other publications cited herein are incorporated by reference in their entirety. In the event that there is a plurality of definitions for terms cited herein, those in this section prevail unless otherwise stated.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about”, whether or not the term is explicitly used, unless explicitly stated otherwise. As used herein, the singular forms “a,” “an,” and “the” include plural referents unless the context clearly dictates otherwise.

As used herein, “alkyl” means a branched, or straight chain chemical group containing only carbon and hydrogen, such as methyl, ethyl, n-propyl, iso-propyl, n-butyl, iso-butyl, sec-butyl, tert-butyl, n-pentyl, iso-pentyl, sec-pentyl, and neo-pentyl. Typically, alkyl groups will comprise 1 to 6 carbon atoms (e.g., 1 to 4, or 1 to 2 carbon atoms).

In some embodiments, the compounds provided herein, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds are routine in the art.

The term “contacting” means bringing at least two moieties together, whether in an in vitro system or an in vivo system.

The expression “effective amount,” when used to describe an amount of compound applied in a method, refers to the amount of a compound that achieves the desired pharmacological effect or other effect, for example an amount that inhibits the abnormal growth or proliferation, or induces apoptosis of cancer cells, resulting in a useful effect.

A “therapeutically effective amount” of a conjugate with respect to the patient method of treatment, refers to an amount of the conjugate(s) in a preparation which, when administered as part of a desired dosage regimen (to a patient, e.g., a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

As used herein, the term “treating” or “treatment” includes reversing, reducing, or arresting the symptoms, clinical signs, and underlying pathology of a condition in manner to improve or stabilize a patient's condition.

As used herein, “patient” (as in the subject of the treatment) means both mammals and non-mammals Mammals include, for example, humans; non-human primates, e.g. apes and monkeys; cattle; horses; sheep; rats; mice; pigs; and goats. Non-mammals include, for example, fish and birds. The term does not require that the subject be under the immediate supervision of a medical professional.

As used herein, “administration” refers to delivery of a compound provided herein by any external route, including, without limitation, IV, intramuscular, SC, intranasal, inhalation, transdermal, oral, rectal, sublingual, and parenteral administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) (“Preparative LC-MS Purification: Improved Compound Specific Method Optimization” K. F. Blom, et al., J. Combi. Chem. 6(6), 874 (2004), which is incorporated herein by reference in its entirety) and normal phase silica chromatography.

### Compounds

Provided herein are compounds which are useful for the treatment of hypertension and hypertensive end stage renal disease (ESRD).

In some embodiments, a compound of Formula (I), or a pharmaceutically acceptable salt thereof, is provided.

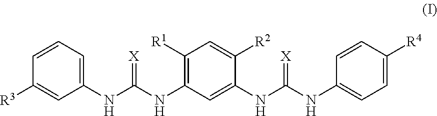

or a pharmaceutically acceptable salt thereof,
wherein:
each X is independently selected from the group consisting of: O and S;
R¹ and R² are independently selected from the group consisting of: H, (C₁-C₆)alkyl, and —C(O)NH(C₁-C₆)alkyl;
R³ is selected from the group consisting of:

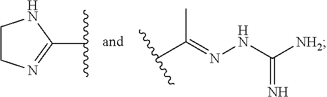

and
R⁴ is selected from the group consisting of:

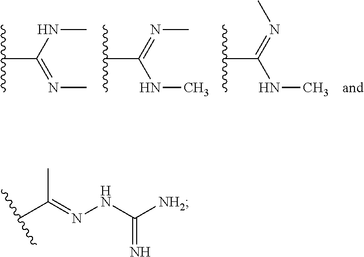

wherein R³ and R⁴ are not the same.

In some embodiments, R₁ and R₂ are independently selected from the group consisting of: H, CH₃, and —C(O)NH(CH₃). In some embodiments, X is O.

In some embodiments, R³ is selected from the group consisting of:

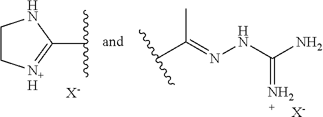

wherein X⁻ is a counteranion. In some embodiments, R³ is

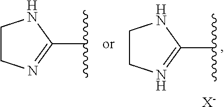

wherein X⁻ is a counteranion.

In some embodiments, R⁴ is selected from the group consisting of:

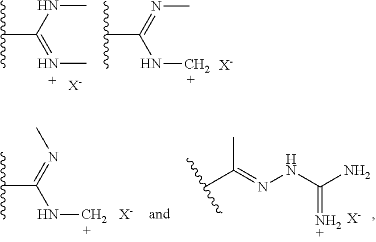

wherein X⁻ is a counteranion. In some embodiments, R⁴ is selected from the group consisting of:

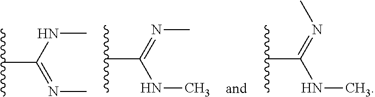

In some embodiments, R⁴ is selected from the group consisting of:

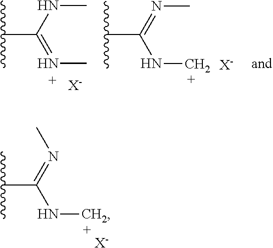

wherein X⁻ is a counteranion.

Non-limiting examples of a compound of Formula (I) include:

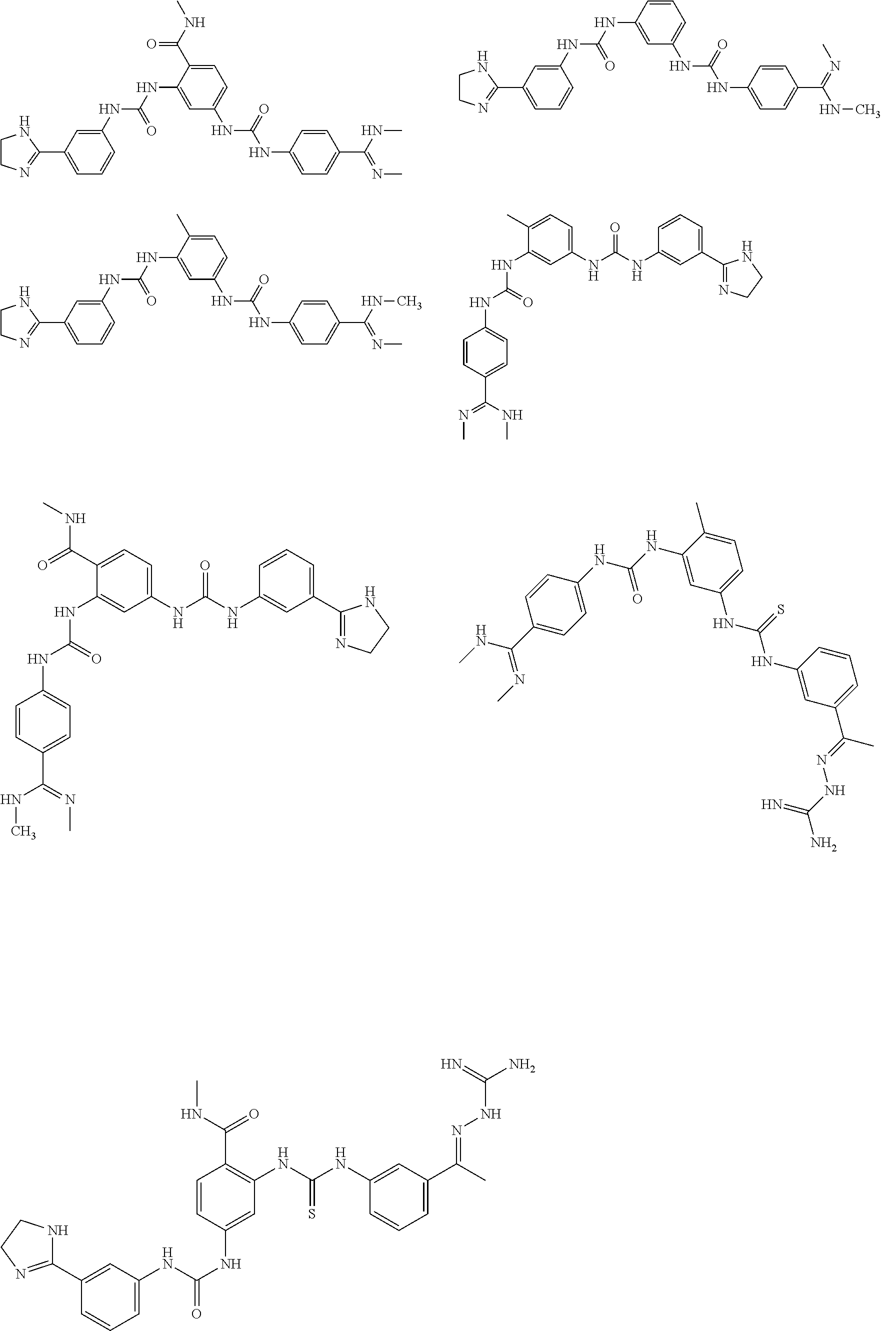

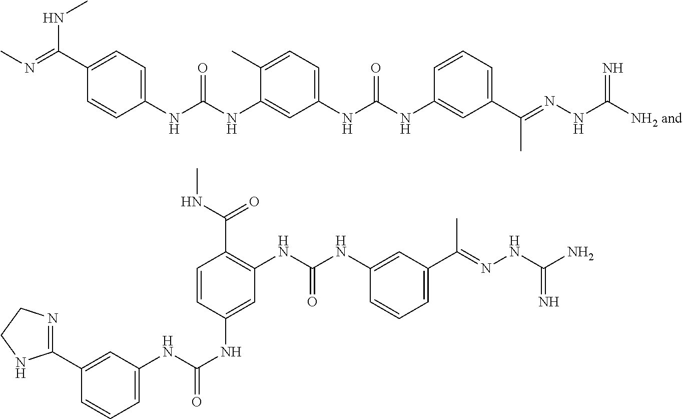

or a pharmaceutically acceptable salt form thereof

For example, a compound of Formula (I) can be selected from:

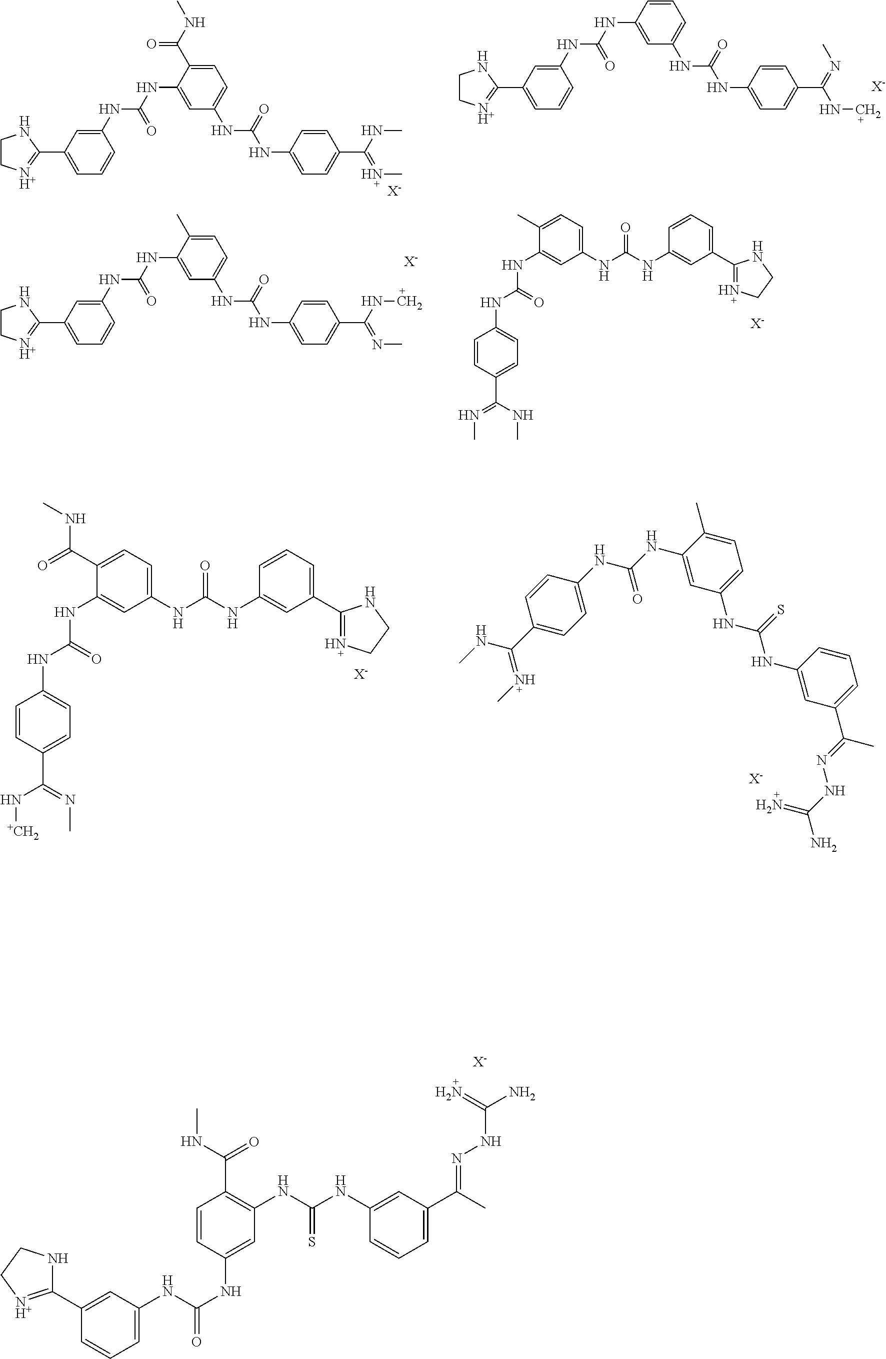

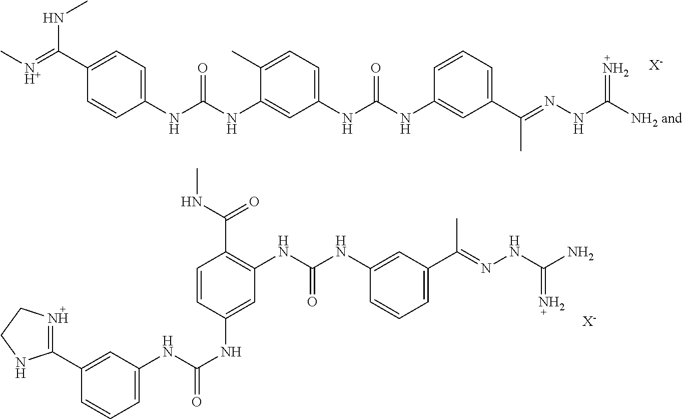

wherein each X⁻ is independently one or more counteranions.

Also provided herein are compounds Formula (II):

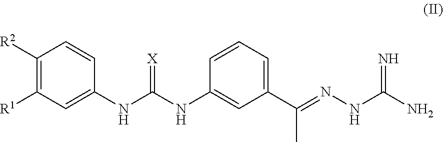

or a pharmaceutically acceptable salt thereof,
wherein:
X is selected from the group consisting of O and S;
R¹ is selected from the group consisting of: H and

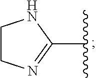 [SMILES: *C1=NCCN1]

R² is selected from the group consisting of: H,

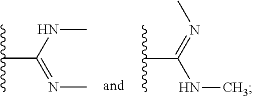

wherein no more than one of R¹ and R² is H.

In some embodiments, a compound of Formula (II) is a compound of Formula (IIA):

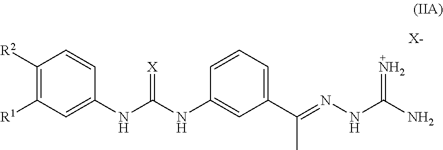

or a pharmaceutically acceptable salt thereof, wherein X⁻ is a counteranion.

In some embodiments, X is S. In some embodiments, X is O.

In some embodiments, R¹ is selected from the group consisting of: H and

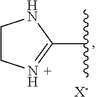

wherein X⁻ is a counteranion.

In some embodiments, R² is selected from the group consisting of: H,

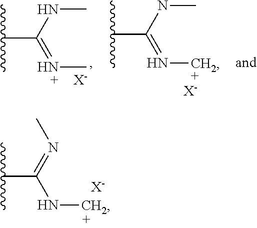

wherein X⁻ is a counteranion.

In some embodiments, R¹ is

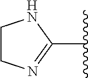 [SMILES: *C1=NCCN1]

and R² is H. In some embodiments, R¹ is

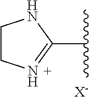 [SMILES: *C1=NCCN1 ; "X-"]

and R² is H, wherein X⁻ is a counteranion. In some embodiments, R¹ is H and R² is

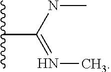 [SMILES: */C(=N/C)NC]

In some embodiments, R¹ is H and R² is

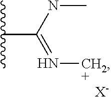

wherein X⁻ is a counteranion.

Non-limiting examples of a compound of Formula (II) include:

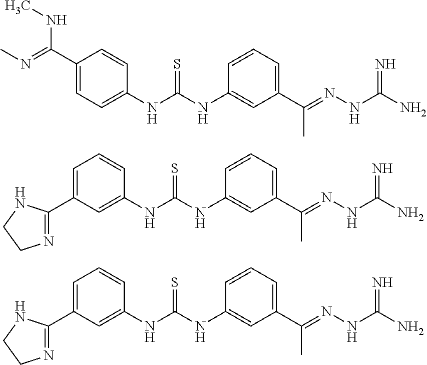 [SMILES: C/N=C(\NC)c1ccc(NC(=S)Nc2cccc(/C(C)=N/NC(=N)N)c2)cc1 ; C/C(=N\NC(=N)N)c1cccc(NC(=S)Nc2cccc(C3=NCCN3)c2)c1 ; C/C(=N\NC(=N)N)c1cccc(NC(=S)Nc2cccc(C3=NCCN3)c2)c1]

or a pharmaceutically acceptable salt thereof.

For example, a compound of Formula (II) can be selected from:

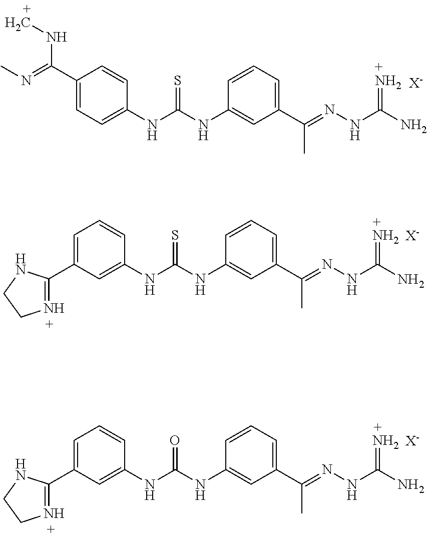

wherein each X⁻ is independently one or more counteranions.

Further provided herein are compound selected from the group consisting of:

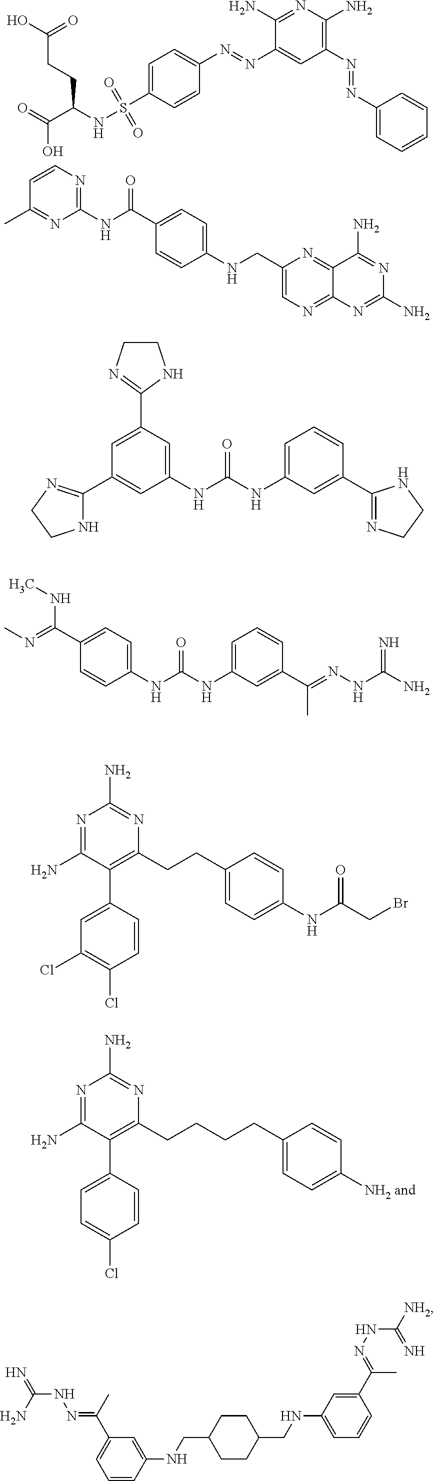

or a pharmaceutically acceptable salt thereof.

In some embodiments, the compound is:

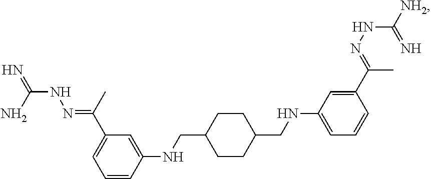 [SMILES: C/C(=N\NC(=N)N)c1cccc(NCC2CCC(CNc3cccc(/C(C)=N/NC(=N)N)c3)CC2)c1]

or a pharmaceutically acceptable salt thereof.

In any of the compounds provided above, each X⁻ represents one or more counteranions. In some embodiments X− is sufficient to provide a net neutral charge for the compound. In some embodiments, two X⁻ are present in each compound (e.g., each X⁻ has a −1 charge). In some embodiments, one X⁻ is present in each compound (e.g., X⁻ has a −2 charge). X⁻ can be any suitable counteranion known by one of ordinary skill in the art. For example, X⁻ can be selected from F⁻, I⁻, Br⁻, Cl⁻, triflate, trifluoroacetate, tosylate, mesylate, mono- or di-carboxylic acid anions having a carbon chain length of between about 1 to 6 (e.g., formate, acetate, propionate etc.), hydrogensulfate, sulfate, phosphate, hydrogenphosphate, dihydrogenphosphate, cyanate, nitrate, sulfide, other anions derived from non-organic acids, and combinations thereof.

In some embodiments, a compound provided herein is an asymmetric diamine compound having a donor on one end, a hydrophobic ring in the middle, and an acceptor on the other end (Cluster A). For example, such a compound can include:

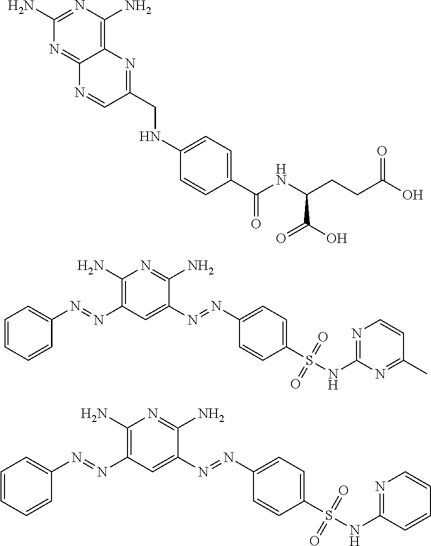 [SMILES: Nc1nc(N)c2nc(CNc3ccc(C(=O)N[C@@H](CCC(=O)O)C(=O)O)cc3)cnc2n1 ; Cc1ccnc(NS(=O)(=O)c2ccc(/N=N/c3cc(/N=N/c4ccccc4)c(N)nc3N)cc2)n1 ; Nc1nc(N)c(/N=N/c2ccc(S(=O)(=O)Nc3ccccn3)cc2)cc1/N=N/c1ccccc1]

or a pharmaceutically acceptable salt thereof.

In other embodiments, a compound is a symmetric dimer with a hydrophobic ring in the middle of the compound and hydrogen-bond donors on both ends (Cluster B). For example, such compounds can be an amidines and may contain a methyl carbamimidoyl phenyl moiety. Exemplary compounds include:

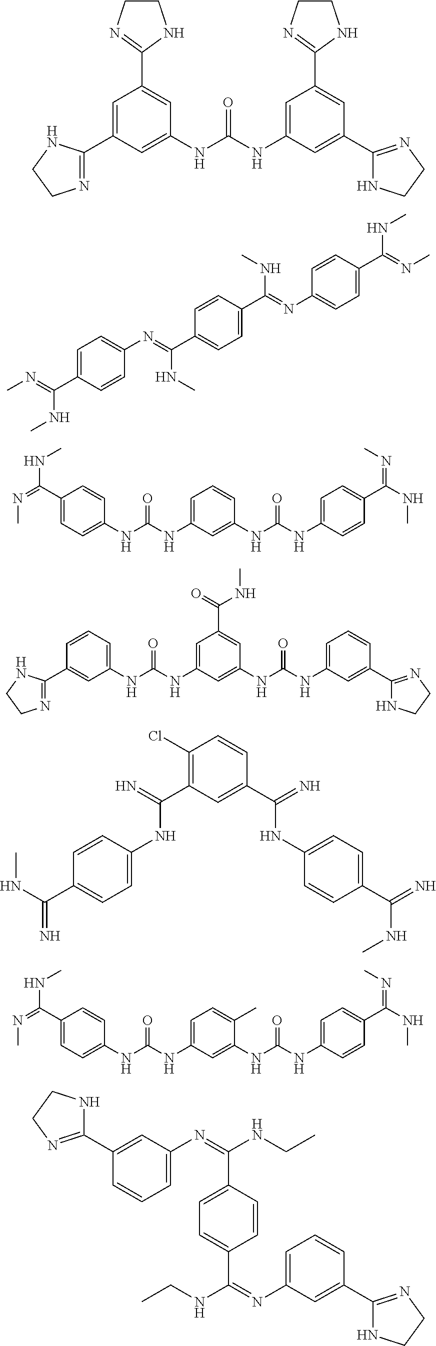

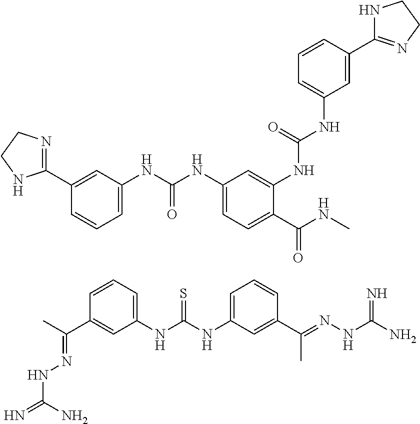 [SMILES: CNC(=O)c1ccc(NC(=O)Nc2cccc(C3=NCCN3)c2)cc1NC(=O)Nc1cccc(C2=NCCN2)c1 ; C/C(=N\NC(=N)N)c1cccc(NC(=S)Nc2cccc(/C(C)=N/NC(=N)N)c2)c1]

or a pharmaceutically acceptable salt thereof.

In some embodiments, a compound provided herein has strong hydrophobic centers on one end, hydrophobic or aromatic rings, and hydrogen-bond donors on the other end (Cluster C). For example, the compound can be an aminopyrimidine. Exemplary compounds include:

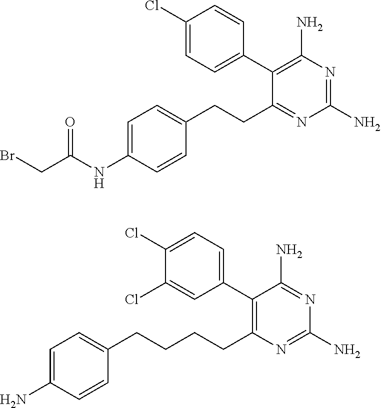 [SMILES: Nc1nc(N)c(-c2ccc(Cl)cc2)c(CCc2ccc(NC(=O)CBr)cc2)n1 ; Nc1ccc(CCCCc2nc(N)nc(N)c2-c2ccc(Cl)c(Cl)c2)cc1]

or a pharmaceutically acceptable salt form thereof.

In other embodiments, a compound provided herein is an asymmetric methylaminopyrimidines with guanidine and dichlorophenyl moieties (Cluster D). For example, such a compound can include:

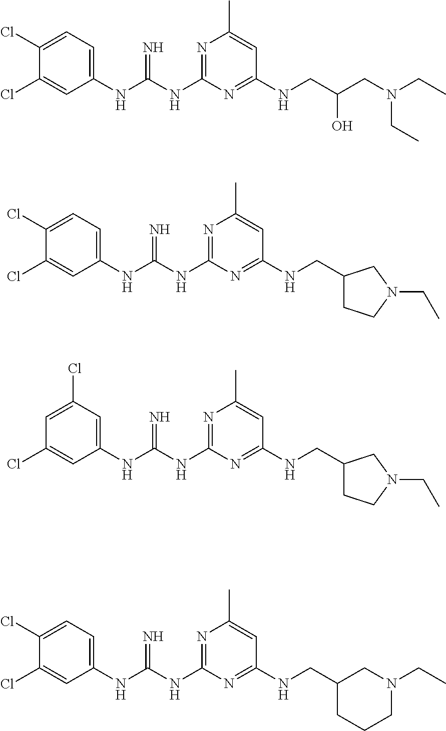 [SMILES: CCN(CC)CC(O)CNc1cc(C)nc(NC(=N)Nc2ccc(Cl)c(Cl)c2)n1 ; CCN1CCC(CNc2cc(C)nc(NC(=N)Nc3ccc(Cl)c(Cl)c3)n2)C1 ; CCN1CCC(CNc2cc(C)nc(NC(=N)Nc3cc(Cl)cc(Cl)c3)n2)C1 ; CCN1CCCC(CNc2cc(C)nc(NC(=N)Nc3ccc(Cl)c(Cl)c3)n2)C1]

or a pharmaceutically acceptable salt thereof.

In some embodiments, a compound provided herein has a hydrophobic center on one end and in the center of the molecule with a donor on the other end (Cluster E). For example, the compound can be a diamino-dimethyl dihydrotriazine. Exemplary compounds include:

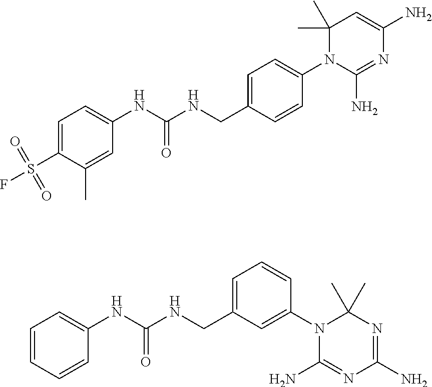 [SMILES: Cc1cc(NC(=O)NCc2ccc(N3C(N)=NC(N)=CC3(C)C)cc2)ccc1S(=O)(=O)F ; CC1(C)N=C(N)N=C(N)N1c1cccc(CNC(=O)Nc2ccccc2)c1]

or a pharmaceutically acceptable salt thereof.

A compound provided herein may be purchased commercially or may be prepared using methods known to those of skill in the art. For example, a compound provided herein can be prepared using a methods as shown in Schemes 1 and 2.

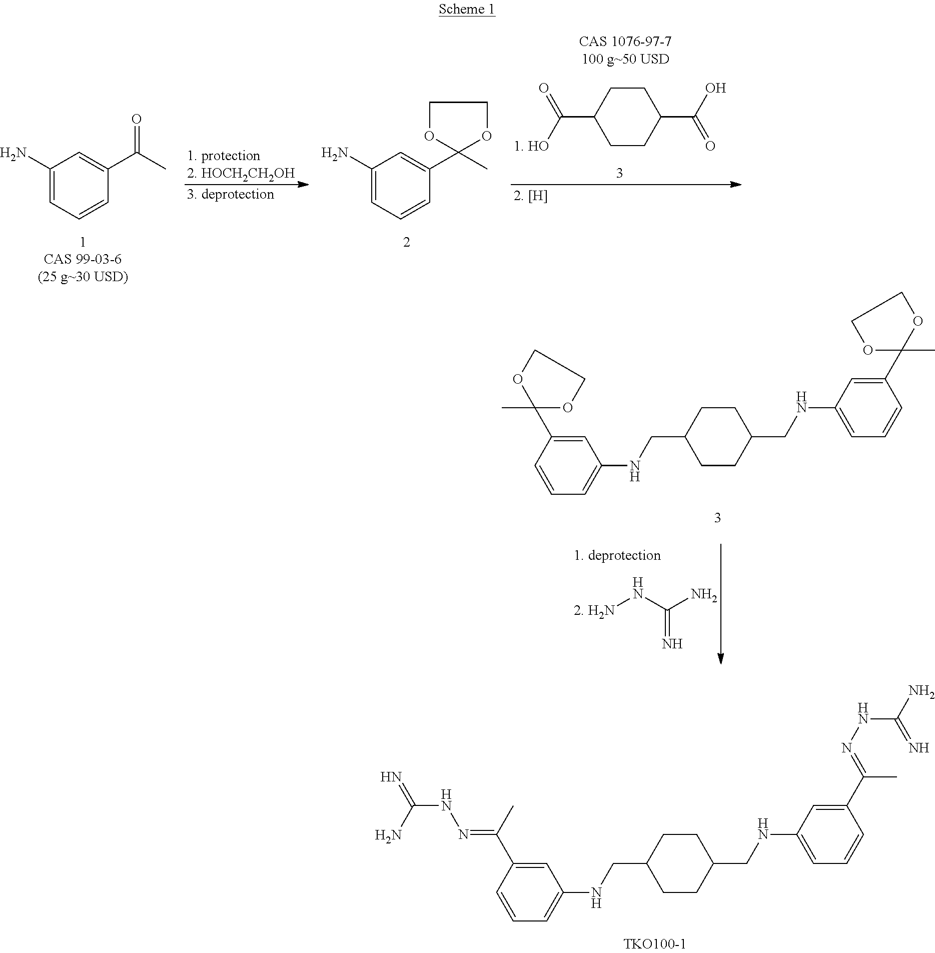

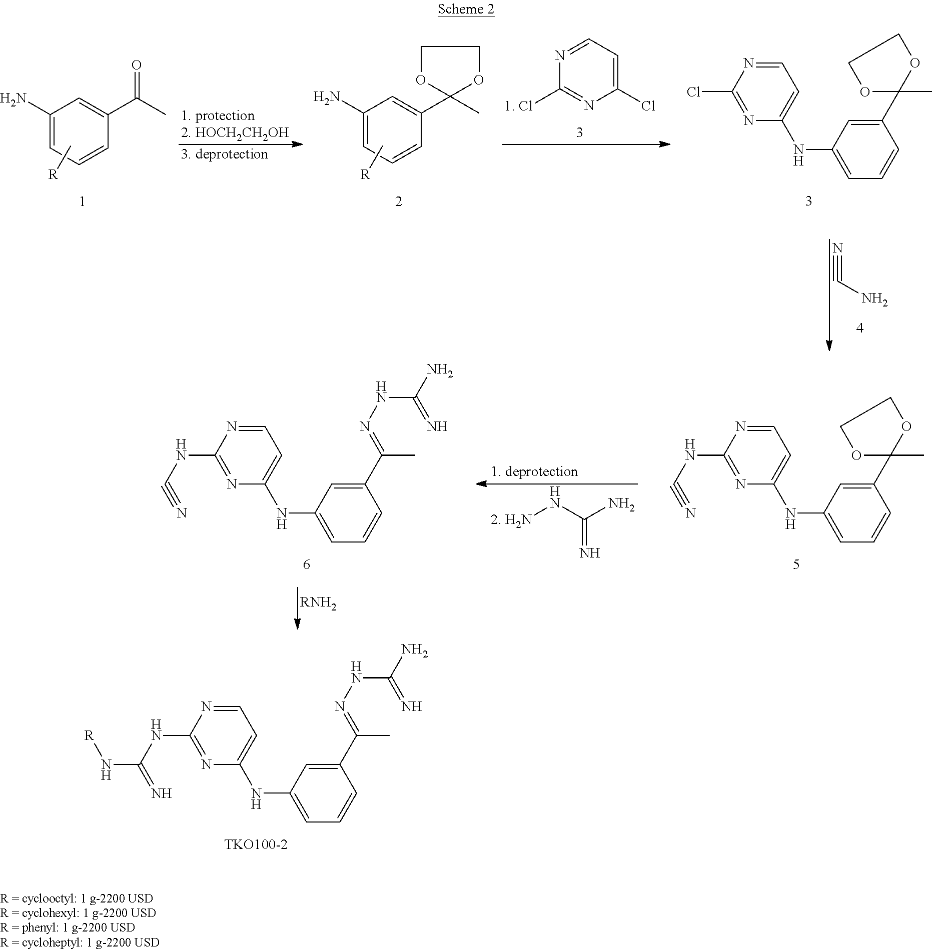

In some embodiments, a compound provided herein can be in the form of a charged species or pharmaceutically acceptable salt form. In other embodiments, a compound provided herein can be in a neutral or uncharged state.

In addition, compounds as provided herein can be identified using screening methods. For example, a compound provided herein can contain at least four pharmacophore annotation points (e.g., at least five pharmacophore annotation points, at least six pharmacophore annotation points). In some embodiments, such pharmacophore annotation points are selected from NCN+ groups or cations, hydrogen bond donors, aromatic or hydrophobic centers, and hydrophobic centers. These pharmacophore annotation points correspond to the chemical nature of those residues of catestatin (CST) believed to be active in binding to AchR, i.e., Leu5, Phe7, Arg8, Arg10, Phe14, and Arg15.

### Methods of Treatment

Disclosed herein, in certain embodiments, are methods of reducing blood pressure, comprising administering to a patient in need thereof a therapeutically effective amount of a compound provided herein. Additionally disclosed herein, in certain embodiments, are methods of treating a disease, disorder or condition characterized by elevated blood pressure, comprising administering to a patient in need thereof a therapeutically effective amount of a compound provided herein. In some embodiments, the disease, disorder or condition characterized by elevated blood pressure is hypertension, pre-hypertension, or hypertensive end stage renal disease (ESRD). In some embodiments, the hypertension is primary hypertension or secondary hypertension. In some embodiments, the hypertension is hypertensive crisis (or, malignant hypertension), pulmonary hypertension, pregnancy-induced hypertension (PIH, or preeclampsia), systolic hypertension, or isolated systolic hypertension.

In some embodiments, a compound provided herein has features such as reduced side effects that make it superior over current methods of treatment. Such side effects can include, for example, cycloplegia, urinary retention, impotence, constipation, syncope, paralytic ileus, and orthostatic hypotension. In some embodiments, a compound provided herein has lower occurrences of reflex tachycardia in patients following administration of the compound compared to pentolinium and trimethaphan, two common treatments for hypertension.

Without being bound by theory, it is believed that the compounds provided herein are non-competitive neuronal nicotinic cholinergic antagonists. When administered in vivo, the compounds provided herein have actions which differ from previous ganglionic blockers: namely, catestatin peptidomimetics reduce heart rate, while hexamethonium (by blocking nicotinic cholinergic activation of parasympathetic tone), by contrast, increases heart rate. This difference results in a difference side effect profile for the compounds provided herein. The effects of catestatin, for example, on histamine release seem to be rather species-specific: present in the rat, but believed to be absent in mouse or human.

The compounds provided herein are also useful as inhibitors (e.g., non-competitive inhibitors) of the nicotinic cholinergic receptors. Accordingly, provided herein, in certain embodiments, are methods of inhibiting a nicotinic cholinergic receptor in a cell. Further, disclosed herein, in certain instances, are methods of treating a disease, disorder or condition characterized by unwanted or excessive activity of nicotinic acetylcholine receptors (nAChRs), comprising administering to a patient in need thereof a therapeutically effective amount of a compound provided herein. In some embodiments, the methods include contacting the cell with an effective amount of a compound provided herein, or a pharmaceutically acceptable salt thereof. The method of inhibiting a nicotinic cholinergic receptor in a cell may be performed by contacting the cell with a compound provided herein, or a pharmaceutically acceptable salt form thereof, in vitro. Uses of such an in vitro method of a nicotinic cholinergic receptor include, but are not limited to use in a screening assay (for example, wherein a compound provided herein is used as a positive control or standard compared to compounds of unknown activity or potency in reducing the amount of one or more nicotinic cholinergic receptors).

The method of inhibiting a nicotinic cholinergic receptor (e.g., a nicotinic acetylcholine receptor (AchR)) in a cell may be performed, for example, by contacting a cell with a compound provided herein, or a pharmaceutically acceptable salt thereof, in vivo, thereby. The contacting is achieved by causing a compound provided herein, or a pharmaceutically acceptable salt form thereof, to be present in a patient in an amount effective to achieve inhibition of one or more nicotinic cholinergic receptors. This may be achieved, for example, by administering an effective amount of a compound provided herein, or a pharmaceutically acceptable salt form thereof, to a patient. Uses of such an in vivo method of inhibiting one or more nicotinic cholinergic receptors include, but are not limited to use in methods of treating a disease or condition, wherein inhibition of one or more nicotinic cholinergic receptors is beneficial.

In some embodiments, a compound provided herein inhibits one or more of the following nicotinic cholinergic receptors: alpha-3, alpha-4, alpha-7, beta-2, beta-4, and combinations thereof. For example, a compound provided herein may inhibit one or more receptor combinations. In some embodiments a compound provided herein inhibits alpha-7, alpha-3 and beta-4; alpha-3 and beta-2; and/or alpha-4 and beta-2.

Inhibition of one or more nicotinic cholinergic receptors can, for example, affect one or more of secretion, transcription, cationic signal transduction, agonist desensitization in a cell.

The activity of the compounds provided herein can be measured using known assays such as those described in the Examples.

### Pharmaceutical Formulations and Dosage Forms

When employed as pharmaceuticals, the compounds provided herein can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal, intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also provides pharmaceutical compositions which contain, as the active ingredient, a compound provided herein or a pharmaceutically acceptable salt thereof, in combination with one or more pharmaceutically acceptable carriers (excipients). In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, an active compound can be milled to provide the appropriate particle size prior to combining with the other ingredients. If an active compound is substantially insoluble, it can be milled to a particle size of less than 200 mesh. If an active compound is substantially water soluble, the particle size can be adjusted by milling to provide a substantially uniform distribution in the formulation, e.g. about 40 mesh.

The compounds provided herein may be milled using known milling procedures such as wet milling to obtain a particle size appropriate for tablet formation and for other formulation types. Finely divided (nanoparticulate) preparations of the compounds provided herein can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions provided herein can be formulated so as to provide quick, sustained or delayed release of the active ingredient after administration to the patient by employing procedures known in the art.

For preparing solid compositions such as tablets, the principal active ingredient is mixed with a pharmaceutical excipient to form a solid preformulation composition containing a homogeneous mixture of a compound provided herein. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient provided herein.

The tablets or pills provided herein can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions provided herein can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

In some embodiments, the compounds provided herein are formulated for intravenous administration. Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound provided herein. The topical formulations can be suitably packaged in tubes of, for example, 100 g which are optionally associated with instructions for the treatment of the select indication.

In one embodiment, the compounds provided herein are prepared with carriers that will protect the compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human patients and other mammals, each unit containing a predetermined quantity of active material calculated to produce the desired therapeutic effect, in association with a suitable pharmaceutical excipient.

In some embodiments, the compositions provided herein contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions provided herein contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions provided herein contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses provided herein.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The therapeutic dosage of a compound provided herein can vary according to, for example, the particular use for which the treatment is made, the manner of administration of the compound, the health and condition of the patient, and the judgment of the prescribing physician. The proportion or concentration of a compound provided herein in a pharmaceutical composition can vary depending upon a number of factors including dosage, chemical characteristics (e.g., hydrophobicity), and the route of administration. For example, the compounds provided herein can be provided in an aqueous physiological buffer solution containing about 0.1 to about 10% w/v of the compound for parenteral administration. Some typical dose ranges are from about 1 mg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### EXAMPLES

### General Methods

Experiments in cultured cells with test compounds were repeated at least 3 times, with 2-3 wells per condition in each experiment. Curve fitting, slopes, and intercepts were computed using Kaleidagraph (Synergy Software, Reading, Pa.). The results were expressed as mean one SEM. Multiple comparisons were made using one-way ANOVA followed by Bonferroni post hoc tests, or by two-way ANOVA using Kaleidagraph. Statistical significance was concluded at p<0.05.

### Example 1

### Design and Molecular Database Screening

The NMR structure of catestatin (CST) (Protein Database ID 1N2Y; N. E. Preece et al., A. Regul. Peptides, 118 (2004), p. 75-87), and the results of alanine scanning of its residues (S. K. Mahata et al., Mol. Endocrinol., 14 (2000), 1525-1535) have been used to design the pharmacophore hypothesis for compounds that would mimic CST binding and actions.

A. Screening Methods

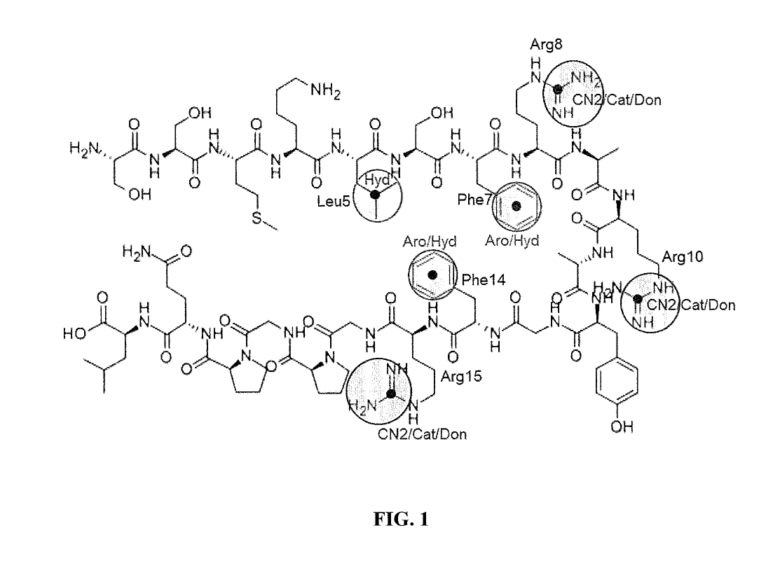

The residues of CST identified as participating in its inhibition of the nicotinic acetylcholine receptor (AchR) were termed its annotation points. A pharmacophore hypothesis was generated relating to these annotation points including atomic, centroid, and bioisosteric annotations. A six-feature pharmacophore model was developed using MOE 2012.10 (Molecular Operating Environment, Chemical Computing Group—CCG, Montreal, Canada). The following pharmacophore features were applied: NCN+ groups or cations or H-bond donors (CN2|Cat|Don), aromatic or hydrophobic centers (Aro|Hyd), and hydrophobic centers (Hyd). Specifically, as shown in FIG. 1, three arginines-Arg8, Arg10, and Arg15—were selected as H-bond donors; two phenylalanines—Phe7 and Phe14—as hydrophobic/aromatic centers; and one leucine—Leu5—as a hydrophobe.

Taking into consideration the possibility that actual binding of CST might be based on as few as two positive-negative contacts (without hydrophobic centers involved), the pharmacophore search was allowed to fit five features of six from the generated pharmacophore. During the screen, various combinations of pairs of NCN+ group/cation/H-bond donor features and aromatic/hydrophobic and hydrophobe features were explored.

Initial compound selection was conducted using the Open NCI Database (http://cactvs.nci.nih.gov/download/nci/) with 3D structures of over 250,000 compounds, and comprehensive searches were conducted against the generated pharmacophore model. Because the generated interface pharmacophore is large—the average distance between centers is 10.82 Å (maximum, 16.28 Å) and the maximum overall length is 19.03 Å—compounds with molecular weights larger than 600 g/mol that are usually used for ligand-based approaches were not excluded; thus a conformational database from the Open NCI Database with all compounds having molecular weights <800 g/mol was created. Four databases with 260,071 compounds having in common 11,762,965 conformations were created. When a five-of-six-feature pharmacophore was searched, 2776 compounds were found with 490,889 conformations. After filtering for configuration and shape, 20 compounds remained (see Table 1).

TABLE 1

| TKO | | | IC₅₀ | | | |
|---|---|---|---|---|---|---|
| # | Structure | CAS RN | (μM) | ASA | SlogP | Cluster |
| 10-1 | 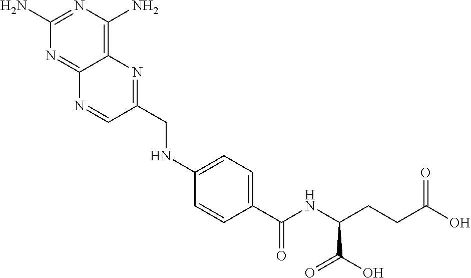 [SMILES: Nc1nc(N)c2nc(CNc3ccc(C(=O)N[C@@H](CCC(=O)O)C(=O)O)cc3)cnc2n1] | 54-62-6 | >5 | 679.04 | −2.16 | A |
| 10-11 | 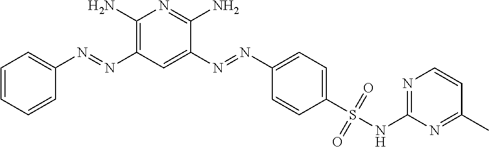 [SMILES: Cc1ccnc(NS(=O)(=O)c2ccc(/N=N/c3cc(/N=N/c4ccccc4)c(N)nc3N)cc2)n1] | 29978-69-6 | >5 | 744.35 | 4.98 | A |
| 10-12 | 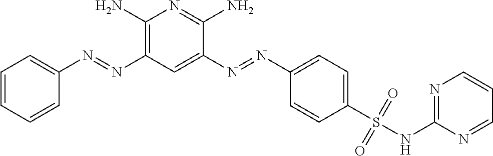 [SMILES: Nc1nc(N)c(/N=N/c2ccc(S(=O)(=O)Nc3ncccn3)cc2)cc1/N=N/c1ccccc1] | 903637-18-3 | 2.0 | 717.14 | 5.27 | A |
| 10-2 | 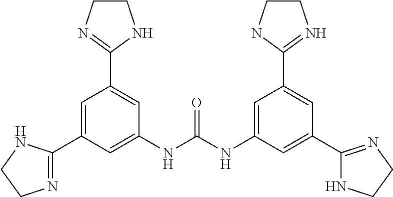 [SMILES: O=C(Nc1cc(C2=NCCN2)cc(C2=NCCN2)c1)Nc1cc(C2=NCCN2)cc(C2=NCCN2)c1] | 5611-46-1 | >5 | 775.32 | −6.95 | B |
| 10-3 | 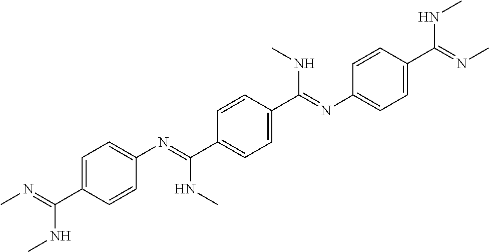 | 13725-66-1 | 1.7 | 789.09 | −2.12 | B |
| 10-4 | 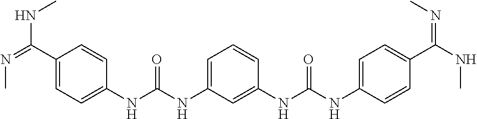 [SMILES: C/N=C(/NC)c1ccc(NC(=O)Nc2cccc(NC(=O)Nc3ccc(/C(=N\C)NC)cc3)c2)cc1] | 5262-16-8 | 0.23 | 805.37 | 2.41 | B |
| 10-5 | 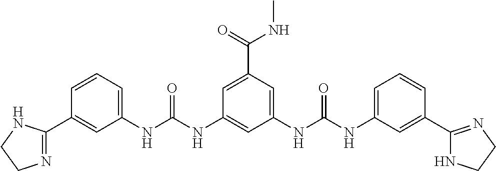 [SMILES: CNC(=O)c1cc(NC(=O)Nc2cccc(C3=NCCN3)c2)cc(NC(=O)Nc2cccc(C3=NCCN3)c2)c1] | 5300-67-4 | 1.1 | 865.73 | −0.80 | B |
| 10-6 | 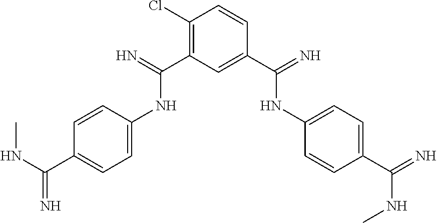 [SMILES: CNC(=N)c1ccc(NC(=N)c2ccc(Cl)c(C(=N)Nc3ccc(C(=N)NC)cc3)c2)cc1] | 13474-78-7 | 1.8 | 761.70 | −0.12 | B |
| 10-7 | 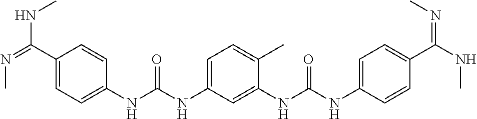 [SMILES: C/N=C(/NC)c1ccc(NC(=O)Nc2ccc(C)c(NC(=O)Nc3ccc(/C(=N\C)NC)cc3)c2)cc1] | 25775-76-2 | 0.29 | 834.56 | −2.72 | B |
| 10-8 | 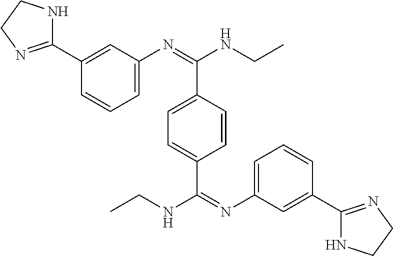 [SMILES: CCN/C(=N/c1cccc(C2=NCCN2)c1)c1ccc(/C(=N\c2cccc(C3=NCCN3)c2)NCC)cc1] | 902261-37-4 | 1.6 | 707.06 | −3.92 | B |
| 10-9 | 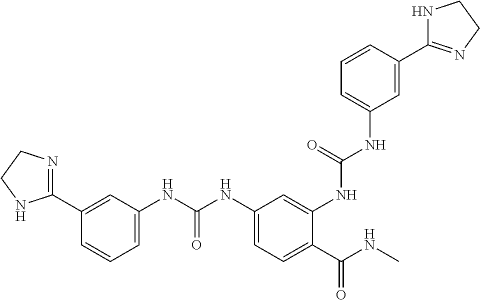 [SMILES: CNC(=O)c1ccc(NC(=O)Nc2cccc(C3=NCCN3)c2)cc1NC(=O)Nc1cccc(C2=NCCN2)c1] | 1135125-97-1 | 1.4 | 852.07 | −0.80 | B |
| 10-18 | 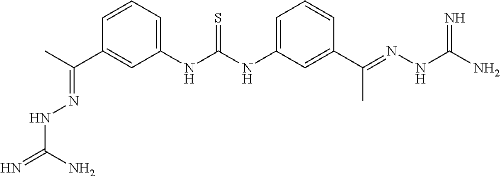 [SMILES: C/C(=N\NC(=N)N)c1cccc(NC(=S)Nc2cccc(/C(C)=N/NC(=N)N)c2)c1] | 20782-52-9 | 0.18 | 700.17 | −1.73 | B |
| 10-10 | 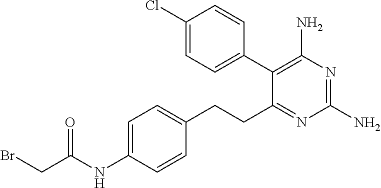 [SMILES: Nc1nc(N)c(-c2ccc(Cl)cc2)c(CCc2ccc(NC(=O)CBr)cc2)n1] | 17005-25-3 | 1.5 | 655.17 | 4.08 | C |
| 10-19 | 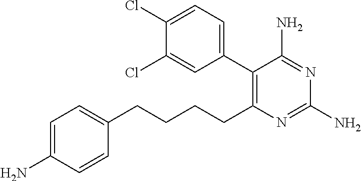 [SMILES: Nc1ccc(CCCCc2nc(N)nc(N)c2-c2ccc(Cl)c(Cl)c2)cc1] | 501678-85-9 | 2.6 | 628.86 | 4.76 | C |
| 10-14 | 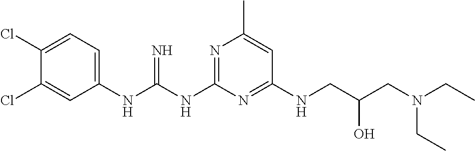 [SMILES: CCN(CC)CC(O)CNc1cc(C)nc(NC(=N)Nc2ccc(Cl)c(Cl)c2)n1] | 51386-92-6 | 0.68 | 733.37 | −0.27 | D |
| 10-16 | 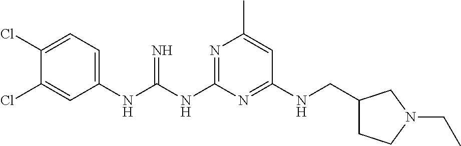 [SMILES: CCN1CCC(CNc2cc(C)nc(NC(=N)Nc3ccc(Cl)c(Cl)c3)n2)C1] | 51387-17-8 | 0.45 | 703.13 | 1.08 | D |
| 10-17 | 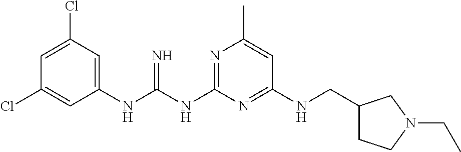 [SMILES: CCN1CCC(CNc2cc(C)nc(NC(=N)Nc3cc(Cl)cc(Cl)c3)n2)C1] | 51387-52-1 | 1.9 | 707.13 | 1.07 | D |
| 10-20 | 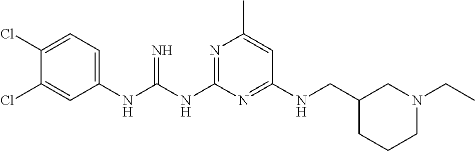 [SMILES: CCN1CCCC(CNc2cc(C)nc(NC(=N)Nc3ccc(Cl)c(Cl)c3)n2)C1] | 21062-28-2 | 1.1 | 687.29 | 1.21 | D |
| 10-13 | 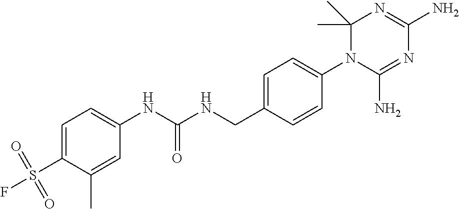 [SMILES: Cc1cc(NC(=O)NCc2ccc(N3C(N)=NC(N)=NC3(C)C)cc2)ccc1S(=O)(=O)F] | 21322-73-6 | 0.87 | 682.11 | −1.41 | E |
| 10-15 | 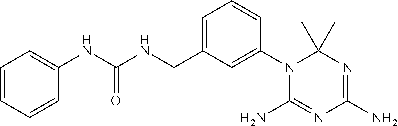 [SMILES: CC1(C)N=C(N)N=C(N)N1c1cccc(CNC(=O)Nc2ccccc2)c1] | 50699-32-6 | >5 | 597.08 | −1.38 | E |

IC₅₀: Concentration that achieves 50% inhibition of secretion; values emerge from the experiments reported herein.
ASA: water-accessible surface area.
SlogP: based-on-structure calculation model of octanol-water partition coefficient P. T.J. Hou et al. J. Chem. Inf. Comput. Sci., 43 (2003), 2137-2152.

B. Similarity Clustering

The similarity clustering method separates molecules by their molecular fingerprints. Molecular fingerprint is a unique ID of the molecule that represents a set of features derived from the its structure. The particular features calculated from a structure and its number can be arbitrary; this difference accounts for the diversification of fingerprints. The most popular fingerprint is a MACCS (Molecular ACCess System) key that is used in many public and commercial databases. The MACCS key encodes molecular structure in a series of numbers that represents the presence or absence of particular substructures in the molecule. There is a binary version of MACCS, BIT_MACCS, which is the same code in binary units (0 or 1). In MOE2012, several fingerprints may be calculated: eigenvalue shape fingerprints, pharmacophore fingerprints, and others.

For similarity clustering, the BIT_MACCS, MACCS, and GpiDAPH3 fingerprints were used. The GpiDAPH3 fingerprint is derived from a 2D molecular graph of a three-point pharmacophore. Each atom is given one of eight atom types calculated from three atomic properties: “in π system,” “is a donor,” and “is an acceptor.” Then all triplets of atoms are coded as features.

After selecting and computing a fingerprint for each of the identified structures, a nearest-neighbor Jarvis-Patrick clustering (R. A. Jarvis, et al. IEEE Trans. Comput., C22 (1973), 1025-1034) with both similarity (S) and overlap (O) parameters—SO was applied, using the Tanimoto coefficient as a similarity metric. The Tanimoto coefficient T(i,j) (P. Willett. J. Chem. Inf. Comput. Sci., 38 (1998), 983-996) estimates similarities between pairs of compounds. For two compounds i and j with fingerprints of length lᵢ and lⱼ, respectively:

T(i,j)=l_{i,j}/(lᵢ+lⱼ−l_{i,j}),

where lᵢ is the number of bits in molecule i, lⱼ is the number of bits in molecule j, and l_{i,j} is the number of common bits between i and j. The similarity scores between the reference molecule and each molecule in the database were computed and ranked, creating clusters.

The following fingerprints and SO values were used: (1) the feature list version MACCS Structural Keys (FP:MACCS), SO=0.55; (2) the binary list version MACCS Structural Keys (FP:BIT_MACCS), SO=0.55; and a three-point-pharmacophore-based fingerprint calculated from 2D molecular graphs (FP: GpiDAPH3, Graph of pi-system-donor-acceptor-polar-hydrophobe three-point pharmacophore), SO=0.45 (C. Williams. Mol. Diversity, 10 (2006), 311-322; and V. L. Kouznetsova et al., Bioorg. Med. Chem. 19 (2011), 3320-3340). These SO values have been shown to be useful for corresponding fingerprint schemes. Previous studies have shown that the reliable clustering can be derived from the GpiDAPH3 fingerprint.

The program created five classes or clusters (A-E) populated by two compounds or more, as well as three single compounds that did not show biological activity (not shown). Table 1 shows the representative compounds selected for these clusters.

The clusters included the following kinds of compounds:

Cluster A.

Three diamine compounds that are asymmetric, have in common donors on one end, a hydrophobic ring in the middle, and acceptors on the other side.

Cluster B.

Nine compounds that are symmetric dimers with the hydrophobic rings in the middle, having hydrogen-bond (H-bond) donors on both ends of the molecule. Additional H-bond acceptors in two of these molecules do not change the overall “positive” character of the molecules in this cluster that corresponds to the donor-hydrophobic pharmacophore profile. They belong to the class of amidines, most containing a methyl carbamimidoyl phenyl moiety.

Cluster C.

Two compounds in this cluster have strong hydrophobe centers on one end, hydrophobic/aromatic rings, and H-bond donors on the other end. Both compounds are aminopyrimidines.

Cluster D.

Five asymmetric compounds that have in common donors—on one end and in the center of the molecule, and hydrophobes on the other end. These compounds are methylaminopyrimidines, with guanidine and dichlorophenyl moieties.

Cluster E.

Two compounds that have hydrophobic centers on one end as well as the center, and donors on the other end. These compounds are diamino-dimethyl triazines.

After experimental validation of the compounds (see below), it was determined that most of the active compounds belonged to cluster B, which included nine active compounds, with three displaying the highest activity in the whole sample set. Cluster A had two active and one inactive compound, while clusters C and E had active compounds of good and very good activity, respectively. Cluster D contained compounds with very good activity.

### Example 2

### Nicotinic Cholinergic Catecholamine Secretion Pathway

Rat PC12 pheochromocytoma cells were grown at 37° C. with 6% CO₂, in 12-well plates, in Dulbecco's modified Eagle's medium (high glucose) supplemented with fetal bovine serum, horse serum, and penicillin/streptomycin. Secretion assays were performed as described previously (N. Biswas, et al., Endocrinology, 149 (2008), 749-757). Cells were labeled for 3 h with 1 μCi/mL of [³H]-norepinephrine (NET678, Perkin-Elmer, Waltham, Mass.), then washed three times with basal medium and one time with secretion buffer. Subsequently, the cells were incubated for 30 min with or without the agonist nicotine (30 μM), in the presence or absence of test compounds (0.5 μM or 5 μM). For IC₅₀ determinations, secretion assays were performed in the presence of increasing concentrations of test compounds. The extracellular secretion medium and the cell lysates were collected for [³H]-norepinephrine measurement by liquid scintillation counting. Results were expressed as percent secretion:

% secretion=[amount released/(amount released+amount in lysate)]×100.

Net secretion was calculated as agonist-stimulated release minus basal release. To establish the specificity of secretory inhibition by test compounds, in some experiments catecholamine release was triggered with other classes of secretagogues that bypass the nicotinic cholinergic pathway: membrane depolarization (55 mM KCl); P2x purinergic receptor stimulation (100 μM ATP); K⁺ (repolarizing) channel blockade (2 mM BaCl₂ in the absence of extracellular Ca²⁺); or a Ca²⁺ ionophore (1 μM ionomycin).

Actions of test compounds were compared with the classical noncompetitive nicotinic antagonist chlorisondamine, wherein labeled cells were incubated with different doses of nicotine in the presence or absence of peptide antagonists or chlorisondamine (5 μM).

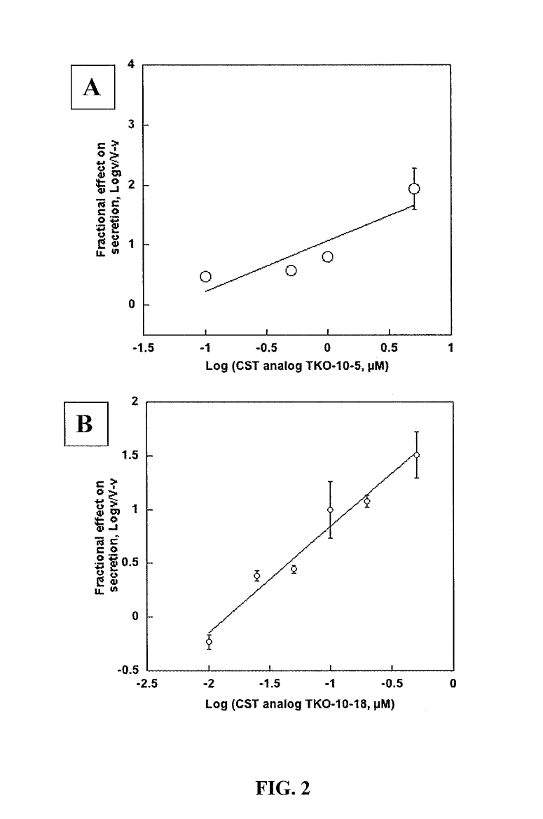

Initial compound screening at 0.5 μM and 5 μM doses established the potencies of TKO-10-4, TKO-10-5, TKO-10-7, and TKO-10-18 from cluster B; TKO-10-14 and TKO-10-16 from cluster D; and TKO-10-13 from cluster E, (see Table 1) with IC₅₀ 1 μM. Human catestatin's IC₅₀ value was previously measured to be 0.82 μM (S. K. Mahata et al., Mol. Pharmacol., 66 (2004), 1180-1191). Secretion assays with increasing compound doses determined the following IC₅₀ values: 0.041 μM (TKO-10-4), 1 μM (TKO-10-5), 0.024 μM (TKO-10-7), and 0.017 μM (TKO-10-18). To test a role for cooperativity in test compound inhibition of secretion, the fractional effect of the compound to inhibit nicotine-stimulated catecholamine secretion was examined as a function of log₁₀ of drug dose (FIGS. 2A and 2B). For compounds TKO-10-5 (FIG. 2A) and TKO-10-18 (FIG. 2B), the plots were linear over a wide range of concentrations, and the Hill slopes were near unity, indicating noncooperative action in each case.

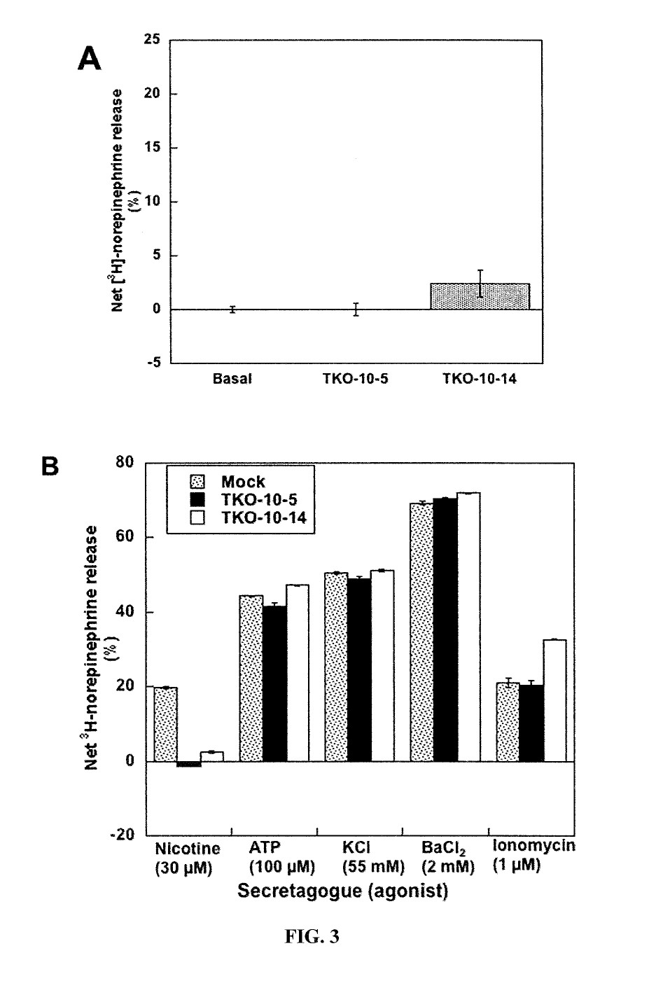

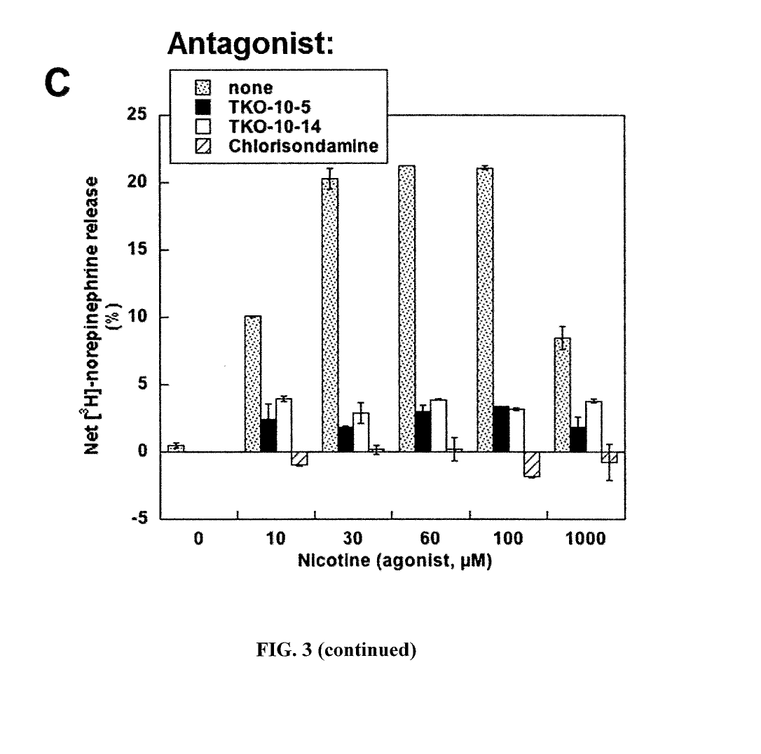

The compounds were also tested to determine their effect on basal secretion. Basal secretion (˜5% ³H-NE release) remained unaltered in presence of TKO-10-5 or TKO-10-14 (FIG. 3A).

In addition to nicotinic-cholinergic stimulation, several secretagogues that act at later stages in the secretory pathway than the nicotinic receptor were tested, including membrane depolarization (by KCl), or blockade of repolarization (by Ba²⁺). In addition, secretagogues that act on different targets from the nicotinic-cholinergic receptor, such as P2x (ATP) or artificial Ca²⁺ pores (Ca²⁺ ionophore ionomycin) were used. Test compounds TKO-10-5 and TKO-10-14 significantly inhibited catecholamine release, but only when triggered by nicotine (FIG. 3B) and not when secretion was caused by agents acting distal to the receptor, or on other receptor classes.

To demonstrate the noncompetitive nature of inhibition, PC12 cells were treated with ascending doses of nicotine (10, 30, 60, 100, and 1000 μM) alone or with test compounds or chlorisondamine (an established noncompetitive neuronal nicotinic cholinergic antagonist) for 30 min, after which cells were harvested for measurement of norepinephrine release (FIG. 3C). Even very high doses of nicotine (100-1000 μM) could not overcome compound inhibition of norepinephrine release, functionally establishing the noncompetitive nature of the inhibition. Although TKO-10-5 or TKO-10-14 were not as potent as chlorisondamine to inhibit catecholamine release, both of them reduced norepinephrine secretion significantly at nicotine concentrations of 10 to 1000 μM (FIG. 3C).

### Example 3

### Nicotinic Cholinergic Signal Transduction: ⁴⁵Ca²⁺ Uptake by PC12 Cells

Nicotinic cholinergic secretory responses in the chromaffin cells are triggered by an early influx of calcium. To test the ability of the compounds to interfere at this early stage of signal transduction, PC12 cells were stimulated with nicotine (30 μM) in the presence or absence of test compounds and agonist-mediated uptake of ⁴⁵Ca²⁺ was measured. ⁴⁵Ca²⁺ uptake was performed as described previously with minor modifications S. K. Mahata et al., J. Clin. Invest., 100 (1997), 1623-1633). Briefly, PC12 cells were grown on poly-L-lysine coated 6-well culture dishes, and were washed with 1 mL release buffer (150 mmol/L NaCl, 5 mmol/L KCl, 2 mmol/L CaCl₂, 10 mmol/L Hepes buffer, pH 7.4) every 15 minutes for 1 hour at 37° C. Cells were then pre-incubated for 1 min in release buffer in the presence or absence of test compounds. Cells were incubated for 2 min with 1 mL of Ca²⁺-free release buffer containing 2 μCi of ⁴⁵Ca²⁺ (catalog number NEZ01300, Perkin-Elmer), 30 μM nicotine in the presence or absence of test compounds. Thereafter, Ca²⁺ uptake was terminated by the addition of 2 mL of ice-cold Ca²⁺-free secretion medium containing 2 mmol/L EGTA and 1 mmol/L LaCl₃, and further washed six times with 2 mL of the same buffer. Cell lysis buffer (secretion medium containing 0.1% Triton X-100, 1 mL) was then added, and the lysate was collected for liquid scintillation counting.

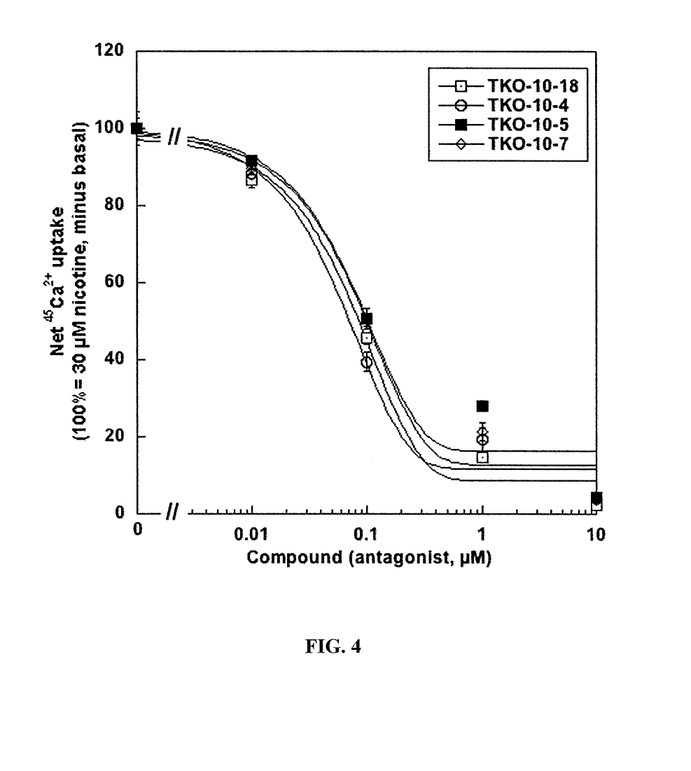

Cluster-B compounds TKO-10-4, TKO-10-5, TKO-10-7, and TKO-10-18 (FIG. 4) inhibited uptake of ⁴⁵Ca²⁺ in a dose-dependent manner, suggesting that the initial interaction with nicotinic receptors remained intact for the compounds. The IC₅₀ (μM) values were ˜0.07 (TKO-10-4), ˜0.1 (TKO-10-5), ˜0.1 (TKO-10-7), and ˜0.09 (TKO-10-18).

### Example 4

### Telemetric Continuous Intra-Arterial Measurement of Blood Pressure in Hypertension

Telemetric measurement of blood pressure (BP) in conscious hypertensive mice (polygenic hypertensive BPH/2J mouse model; F. A. Wright et al., Hypertension, 34 (1999), p. 625-630) was achieved using a Data Sciences International (DSI; Transoma Medical) Physiol Tel telemetry system, implanting a catheter coupled to a TA11PA-C20 (DSI) transmitter, as described (N. R. Mahapatra et al., J. Clin. Invest., 115 (2005), 1942-1952). The animals and implants were left to stabilize for ten days after surgery. BPH/2J polygenic hypertensive mice (n=7/group) were then treated with test compound (5 μg/g body weight, intra-peritoneal; or vehicle). BP and heart rate (HR) were measured continuously over 24 hours, before and after compound administration.

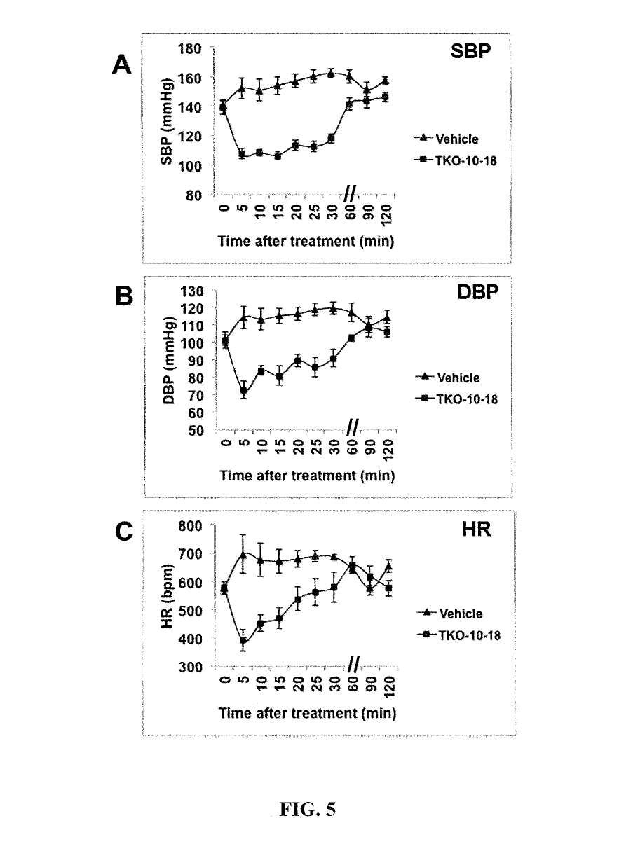

Hypertensive BPH/2J mice (initial SBP/DBP 145.0±0.6/104.9±0.1 mmHg) demonstrated significant reductions in both SBP (by ˜33 mmHg) and DBP (by ˜25 mmHg) after treatment with the TKO-10-18 (though not with vehicle); the declines were apparent by 10-30 min and were maintained for up to ˜60 min (FIGS. 5A and 5B). The fall in BP after the compound was not accompanied by reflex tachycardia; indeed, the HR fell by ˜180 beats/min (FIG. 5C), indicating preserved autonomic and baroreflex function.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound of Formula (I): 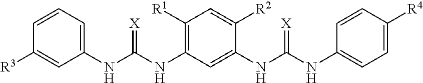
or a pharmaceutically acceptable salt thereof,
wherein:
each X is independently selected from the group consisting of: O and S;
R¹ and R² are independently selected from the group consisting of: H, (C₁-C₆)alkyl, and —C(O)NH(C₁-C₆)alkyl;
R³ is selected from the group consisting of: 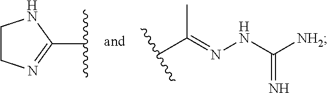
and
R⁴ is selected from the group consisting of: 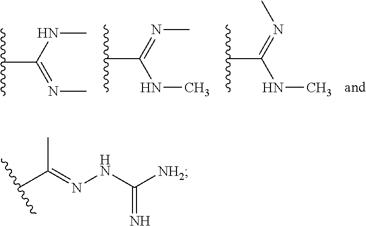
wherein R³ and R⁴ are not the same.

2. The compound of claim 1, wherein R₁ and R₂ are independently selected from the group consisting of: H, CH₃, and —C(O)NH(CH₃).

3. The compound of claim 1, wherein X is O.

4.-5. (canceled)

6. The compound of claim 1, wherein R³ is

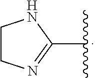 [SMILES: *C1=NCCN1]

7. (canceled)

8. The compound of claim 1, wherein R⁴ is selected from the group consisting of:

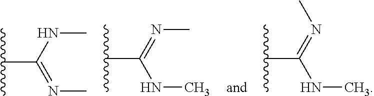

9. (canceled)

10. The compound of claim 1, wherein the compound of Formula (I) is selected from the group consisting of: 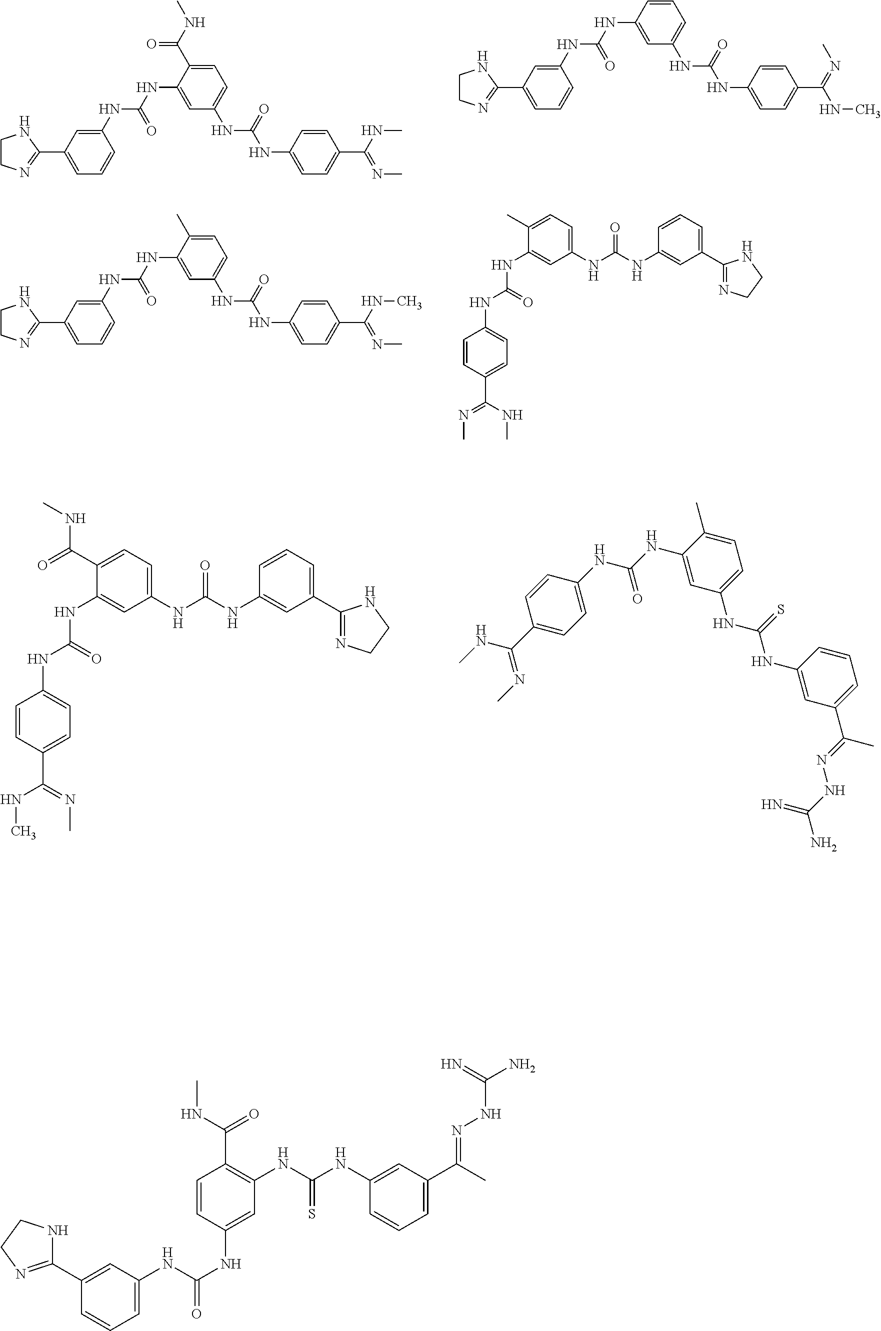 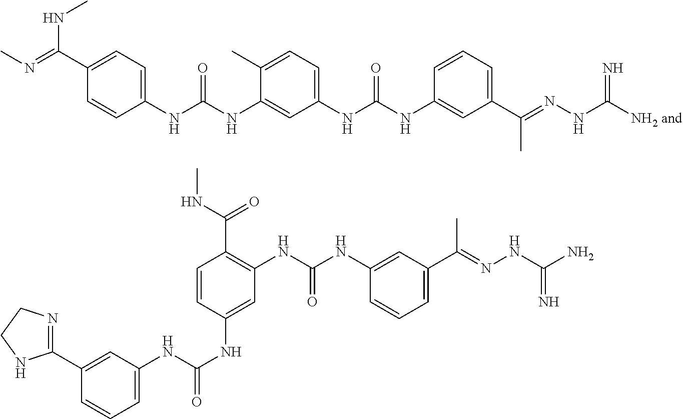
or a pharmaceutically acceptable salt form thereof.

11. (canceled)

12. A compound of Formula (II): 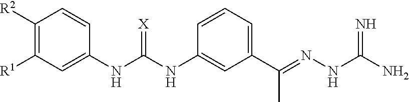
or a pharmaceutically acceptable salt thereof,
wherein:
X is selected from the group consisting of O and S;
R¹ is selected from the group consisting of: H and 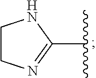 [SMILES: *C1=NCCN1]
R² is selected from the group consisting of: H, 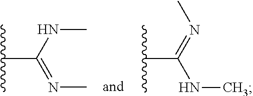
wherein no more than one of R¹ and R² is H.

13. (canceled)

14. The compound of claim 12, wherein X is S.

15. The compound of claim 12, wherein X is O.

16. (canceled)

17. (canceled)

18. The compound of claim 12, wherein R¹ is 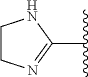 [SMILES: *C1=NCCN1]
and R² is H.

19. (canceled)

20. The compound of claim 12, wherein R¹ is H and R² is

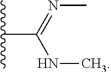 [SMILES: */C(=N/C)NC]

21. (canceled)

22. The compound of claim 12, wherein the compound of Formula (II) is selected from the group consisting of: 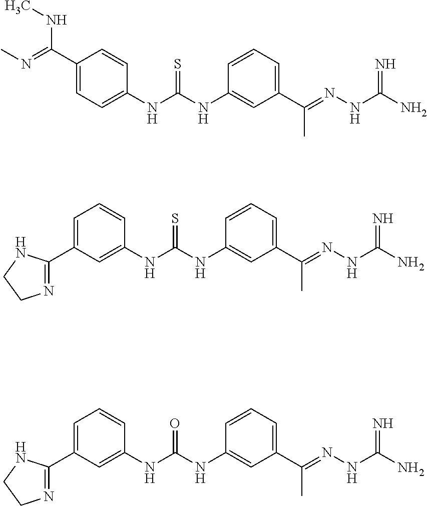 [SMILES: C/N=C(\NC)c1ccc(NC(=S)Nc2cccc(/C(C)=N/NC(=N)N)c2)cc1 ; C/C(=N\NC(=N)N)c1cccc(NC(=S)Nc2cccc(C3=NCCN3)c2)c1 ; C/C(=N\NC(=N)N)c1cccc(NC(=O)Nc2cccc(C3=NCCN3)c2)c1]
or a pharmaceutically acceptable salt thereof.

23.-28. (canceled)

28. A method for treating hypertensive end stage renal disease (ESRD) in a patient in need thereof, the method comprising administering to the patient a therapeutically effective amount of a compound of claim 1, or a pharmaceutically acceptable salt thereof.

29.-38. (canceled)

39. The method of claim 28, wherein the compound is selected from the group consisting of: 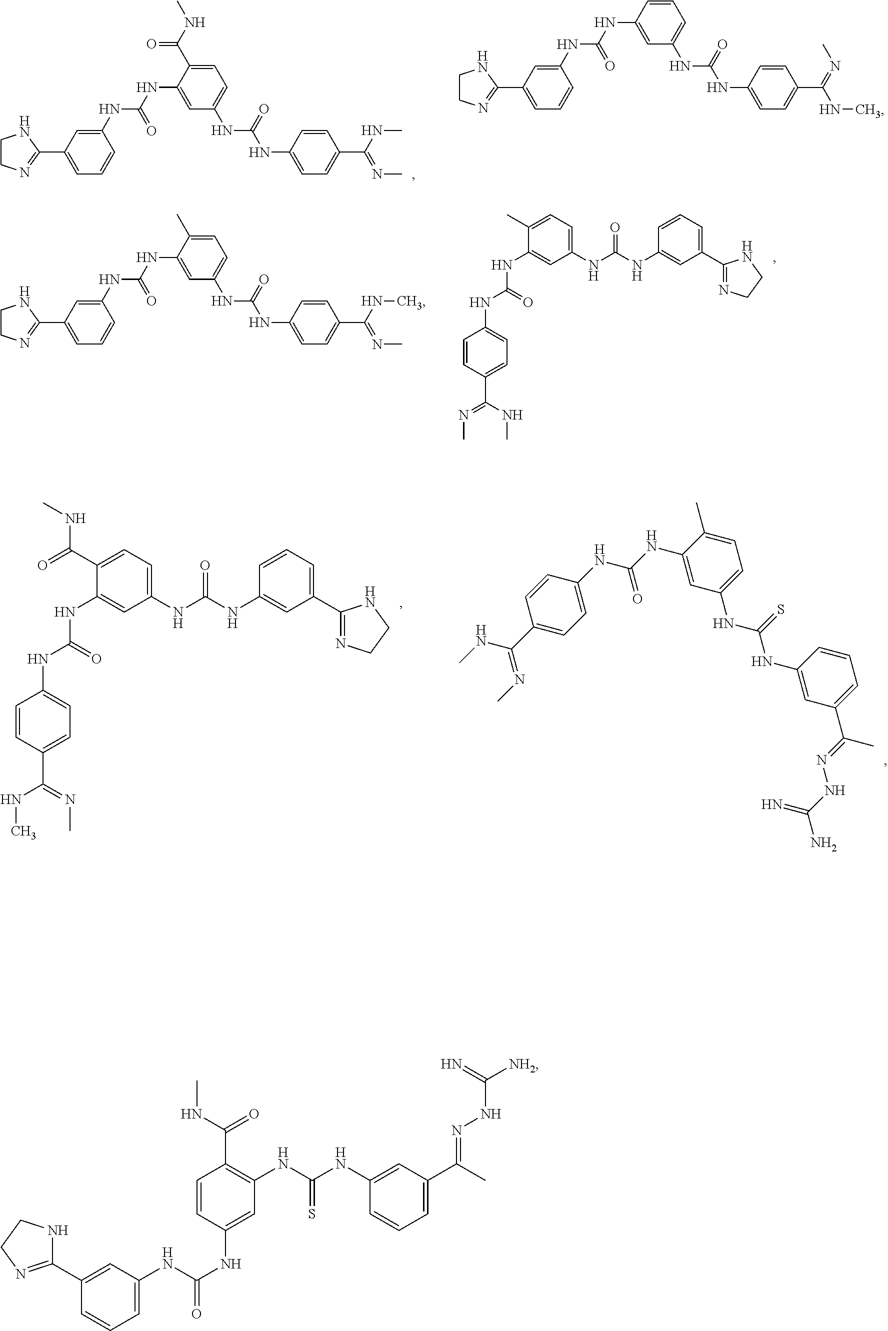 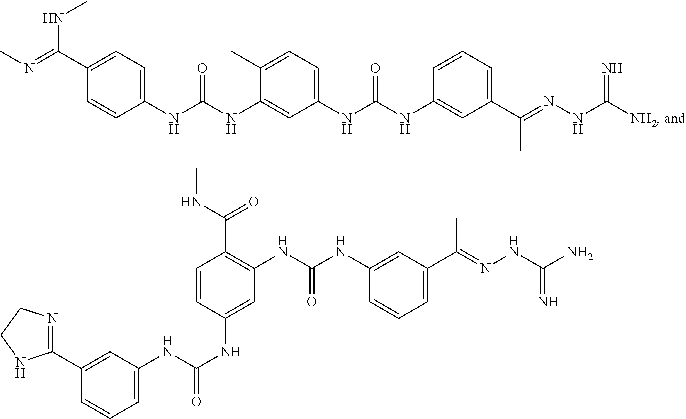
or a pharmaceutically acceptable salt form thereof.

40. A method for treating hypertensive end stage renal disease (ESRD) in a patient in need thereof, the method comprising administering to the patient a therapeutically effective amount of a compound of claim 12, or a pharmaceutically acceptable salt thereof.

41. The method of claim 40, wherein the compound is selected from the group consisting of: 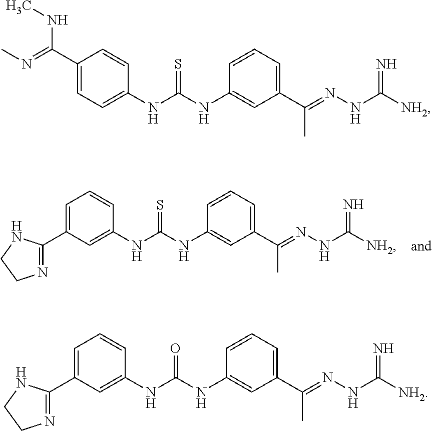 [SMILES: C/N=C(\NC)c1ccc(NC(=S)Nc2cccc(/C(C)=N/NC(=N)N)c2)cc1 ; C/C(=N\NC(=N)N)c1cccc(NC(=S)Nc2cccc(C3=NCCN3)c2)c1 ; C/C(=N\NC(=N)N)c1cccc(NC(=O)Nc2cccc(C3=NCCN3)c2)c1]
